(19) United States
(12) Patent Application Publication
(10) Pub. No.: US 2025/0163357 A1
(43) Pub. Date: May 22, 2025

(54) CAPACITANCE PROBE AND CELL CULTURE FAILURE MODES

(71) Applicant: Regeneron Pharmaceuticals, Inc., Tarrytown, NY (US)

(72) Inventors: Laura O'Brien, Tramore (IE); Hai-Yuan Goh, Limerick (IE)

(21) Appl. No.: 18/953,731

(22) Filed: Nov. 20, 2024

Related U.S. Application Data
(60) Provisional application No. 63/601,636, filed on Nov. 21, 2023.

(51) Int. Cl. C12M 1/34

(52) U.S. Cl. CPC C12M 41/36; C12M 41/12; C12M 41/26; C12M 41/32; C12M 41/34

(57) ABSTRACT

The present invention generally pertains to methods of characterizing a condition of a cell culture. In particular, the present invention pertains to the use of a capacitance probe to derive impedance spectroscopy parameters that correlate to failure modes of a bioprocess.

### CROSS-REFERENCE TO RELATED APPLICATIONS

This application claims the benefit of U.S. Provisional Patent Application No. 63/601,636, filed on Nov. 21, 2023 which is incorporated herein by reference.

### BACKGROUND

Real-time monitoring of recombinant cell culture conditions is essential for responding to deviations in a bioprocess. Online, in-line, at-line, and off-line sensors for bioprocesses are currently used in biopharmaceutical manufacturing, which fulfill varying functions and each have respective limitations. Off-line sensors can measure the true state of a cell culture in terms of its cell density and viability, but cannot do so in real-time. Other sensors can measure cell culture media parameters such as pH, temperature, and dissolved oxygen in real-time, but cannot measure real-time deviations in viable cell density (VCD). Thus, a need exists for real-time monitoring of bioprocesses that can perform early detection of deviations to a cell culture and offer accurate, real-time VCD measurements.

### SUMMARY

Systems and methods have been developed for real-time monitoring of failure modes in a bioprocess. A capacitance probe embedded in a bioreactor may be used to measure permittivity of a cell culture at multiple frequencies and at multiple time points. These measurements can then be used to derive parameters correlating to viable cell density, viable cell volume, cell size, and other cell features. These parameters may be used to detect excursions from desired bioreactor operating conditions, such as temperature changes, pH changes, dissolved oxygen changes, or insufficient or excess nutrient feeds.

This disclosure provides methods for characterizing a condition of a cell culture. In some exemplary aspects, these methods can comprise (a) measuring capacitance of a cell culture using a capacitance probe at two or more frequencies at two or more time points; (b) using the capacitance measurements to calculate at least one impedance spectroscopy model parameter at the two or more time points; and (c) using said at least one model parameter to characterize a condition of said cell culture.

In one aspect, the cell culture comprises CHO cells. In another aspect, the cell culture comprises recombinant cells, wherein said recombinant cells express a protein selected from the group consisting of an antibody, an Fc-fusion protein, a receptor-Fc-fusion protein, and a fragment thereof. In a further aspect, the cell culture comprises recombinant cells expressing Dupilumab.

In one aspect, measuring the capacitance of the cell culture includes measuring the permittivity of the cell culture.

In one aspect, at least one of the two or more time points is from day 0 to day 20 of said cell culture, from day 1 to day 18, from day 3 to day 12, at day 0, at day 1, at day 2, at day 3, at day 4, at day 5, at day 6, at day 7, at day 8, at day 9, at day 10, at day 11, at day 12, at day 13, at day 14, at day 15, at day 16, at day 17, at day 18, at day 19, or at day 20.

In one aspect, at least one of the two or more time points is during a cell culture phase selected from the group consisting of N-5, N-4, N-3, N-2, N-1, and N. In another aspect, at least one of the two or more time points is during a seed train phase of the cell culture. In a further aspect, at least one of the two or more time points is during a production phase of the cell culture.

In one aspect, at least one of the two or more frequencies is about 1 MHz. In another aspect, at least one of the two or more frequencies is about 20 MHz.

In one aspect, calculating the at least one impedance spectroscopy model parameter comprises fitting the measurements to the Cole-Cole model. In another aspect, the at least one impedance spectroscopy model parameter is selected from the group consisting of the characteristic frequency (f_{c}), the alpha parameter (α), and the permittivity increment (Δε).

In one aspect, the capacitance probe is embedded in a bioreactor. In another aspect, the condition is an operating condition of a bioreactor. In a further aspect, the condition is a failure mode. In an additional aspect, the condition is selected from the group consisting of a temperature excursion, an insufficient dextrose feed, an excess dextrose feed, a dissolved oxygen excursion, and a pH excursion. In another aspect, a cell culture may be subjected to one failure mode, two failure modes, or more failure modes.

These, and other, aspects of the invention will be better appreciated and understood when considered in conjunction with the following description and accompanying drawings. The following description, while indicating various aspects and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 (Hamilton Company, 2021, Bioprocess monitoring and control (off-line, at-line, on-line, in-line/in-situ)) illustrates the dual frequency measurement principle for correlating permittivity to real-time viable cell density, according to an exemplary aspect.

FIG. 2 (Hamilton, 2021) illustrates the relationship between permittivity and the impedance spectroscopy parameters f_{c}, Δε, and α, according to an exemplary aspect.

FIG. 3 illustrates a workflow of the experimental design for the Examples, according to an exemplary aspect.

FIG. 4 shows a bivariate fit model throughout the seed train of a bioreactor which combines offline viable cell density values and online permittivity readings obtained using a capacitance probe, according to an exemplary aspect.

FIG. 5 shows calculated real-time viable cell density derived from the measured permittivity value, compared to offline measured viable cell density, according to an exemplary aspect.

FIG. 6 shows a comparison of real-time calculated viable cell density to offline measured viable cell density throughout a whole bioprocess, according to an exemplary aspect.

FIG. 7 shows a comparison of real-time calculated viable cell density to offline measured viable cell density for four bioreactors subjected to varying failure modes, according to an exemplary aspect.

FIG. 8 shows Δε measurements compared to online permittivity values throughout seed train and production phases, according to an exemplary aspect.

FIG. 9A shows the impedance parameter f_{c} throughout the entire bioprocess for four bioreactors subjected to varying failure modes, according to an exemplary aspect.

FIG. 9B shows the impedance parameter Δε throughout the entire bioprocess for four bioreactors subjected to varying failure modes, according to an exemplary aspect.

FIG. 9C shows the impedance parameter α throughout the entire bioprocess for four bioreactors subjected to varying failure modes, according to an exemplary aspect.

FIG. 10A shows the impedance parameter f_{c} at day 1 of a seed train process for four bioreactors, one of which was subjected to a high temperature excursion, according to an exemplary aspect.

FIG. 10B shows the impedance parameter Δε at day 1 of a seed train process for four bioreactors, one of which was subjected to a high temperature excursion, according to an exemplary aspect.

FIG. 10C shows the impedance parameter α at day 1 of a seed train process for four bioreactors, one of which was subjected to a high temperature excursion, according to an exemplary aspect.

FIG. 11A shows the impedance parameter f_{c} for four bioreactors subjected to varying failure modes, according to an exemplary aspect.

FIG. 11B shows the impedance parameter Δε for four bioreactors subjected to varying failure modes, according to an exemplary aspect.

FIG. 11C shows the impedance parameter α for four bioreactors subjected to varying failure modes, according to an exemplary aspect.

### DETAILED DESCRIPTION

Chinese Hamster Ovary (CHO) cells are the current gold standard mammalian host cell line utilized in the bioproduction of recombinant proteins. CHO cells' robustness, such as their ability to withstand the turbulent conditions inside stirred tank bioreactors, as well as the ability to grow in suspension, make them particularly attractive for large scale bioprocessing (Bryan et al., 2021, Current Research in Biotechnology, 3: 49-56). Furthermore, they are a well-characterized platform technology, enabling rapid transfection with the target gene or protein, and successful amplification. Process development using CHO cells has an established history of enabling high yielding products and generating products which demonstrate safety for patients.

The manufacture of biologic medicines may be divided into two distinct subsections: upstream and downstream bioprocessing. The upstream bioprocess initiates with the small-scale expansion of a master batch stock culture, which is a highly characterized starting culture and is produced from the original therapeutic-producing cell line. The master cell bank (MCB) is cryopreserved in small vials, eliminating the potential for genetic discrepancies to occur, and effectively reducing the potential for contamination through minimizing the number of times the cell line is passaged. From the MCB, the working cell banks (WCB) are derived, which are directly involved in the creation of medical products. The starter culture vial is thawed under tightly controlled process conditions and is seeded into a shake flask. Once incubated, the cells are transferred into a wave bag which offers enhanced fluid dynamics enabling optimum mass and gas transfer through the constant exposure of media and oxygen to the cells, promoting growth. Once the desired cell numbers are reached, the process advances to the seed train bioreactors and then on to the production bioreactors under conditions that are optimized to produce the desired recombinant protein.

Harvest is the intermediate stage between the two subsections of the bioprocess, which involves centrifugation and the removal of the bulk contaminants such as cellular debris and particulates. Lastly, downstream processing involves a series of unit operations which aims to isolate, purify and concentrate the target protein to a high degree of purity, removing any cellular debris and performing viral inactivation to ensure the final drug product is safe and efficacious for patient use. Chromatography columns such as affinity chromatography (AC), hydrophobic interaction chromatography (HIC), ion exchange chromatography (IEX) and size exclusion or gel filtration (SEC, GPC) are popular methods for a highly pure end-product. Following concentration of the desired protein using ultrafiltration, the product undergoes extensive characterization prior to final formulation and packaging.

Quality by Design (QbD) is a holistic framework which has reshaped the pharmaceutical and biopharmaceutical industry's previous perception of product quality in recent years. QbD was adopted by the FDA to improve the standards of pharmaceutical manufacturing; from there, it quickly evolved into a comprehensive multidimensional framework emphasizing quality at every stage of the manufacturing process, focusing on the critical quality attributes (CQAs) of the drug, which relates to the chemistry, manufacturing and control (CMC) of the processes and potential implications on the product (Zhang et al., 2016, Drug Discovery Today, 21(5):740-765). Furthermore, this philosophy promotes continuous improvement and encourages innovation throughout the product lifecycle. Quality must be built into the product throughout each stage of the process to ensure it is of the highest acceptable quality for the patients (EMA, 2020, Quality by Design, European Medicines Agency). The QbD philosophy is only feasible using sound scientific principles and quality risk management (QRM).

Biologic medicines are highly complex and variable by their nature, compared to small molecule drugs which are one dimensional chemically synthesized molecular entities. Biologic drug products have multiple stages that can impact the quality and identity of the end-product (Jeffers et al., 2020, Journal of the Advanced Practitioner in Oncology, 11(3):245). Their biologic activity and efficacy are largely influenced by their structure, which is heavily influenced by the manufacturing process. The complexity of these medicines is classified by their physical, chemical, biological, and microbial properties which define them. Such properties are widely known as a drug's quality attributes; however, the attributes that directly impact upon product quality are the CQAs which are of particular significance in biomanufacturing (Vulto and Jaquez, 2017, Rheumatology, 56(suppl_4) iv14-iv29). Thus, the CQAs must be consistently monitored throughout the lifecycle of the product. Validating that the process is in a routine state of control within the specified limits and ranges ensures that the final desired product is obtained on a consistent and routine basis. QbD offers a risk-based approach for designing processes which help to identify a drug's CQAs and setting the limits of these CQAs based on clinical impact to the patient. Building in quality to these complex processes establishes a link between multiple input streams and process parameters which may directly or indirectly affect the quality of the product.

The primary step of QbD in biopharmaceutical production is establishing the quality target product profile (QTPP). The QTPP is a prospective overview of the key quality characteristics that the drug product will achieve to ensure the desired quality is achieved. The target product profile (TPP) is a summary of the overall purpose of the drug development program and provides information regarding the drug at specific timepoints in the development lifecycle. Once established, the QTPP encapsulates information regarding the final drug product from the mode of action, clinical indication, safety profile, route of administration and drug product quality criteria (Zhang et al.). Once the CTPP and CQAs have been established, the next crucial step in the QbD process is to define the CPPs. The CPPs are process parameters which have a direct impact on the CQAs and thus must be closely monitored. The initial monitoring of the CPPs involves utilizing a risk-based approach based on the likelihood of a specific parameter impacting a CQA. To further analyze the severity of the impact and to identify any other parameters that can impact the CQAs, a series of studies must be performed. These studies are commonly known as Design of Experiment studies and can help establish a deeper process understanding and assist in process characterization, which aims to identify key operational and performance parameters to establish an acceptable range for biomanufacturing a high-quality product. Although QbD is not a regulatory requirement, there is no doubt it has greatly improved the consistency, profitability, efficiency and safety of biopharmaceutical drug production and continues to provide a higher level of assurance for product quality, thus reducing the time it takes to obtain regulatory approval.

Encapsulating the holistic framework of QbD, process analytic technology (PAT) enriches the understanding and control of the manufacturing process to achieve a comprehensive QbD, one where quality is built into the product at every stage of the process, and to mitigate risks (Ding et al., 2020). In recent years there have been significant advancements in accurate, real-time monitoring tools in the biopharmaceutical industry, with advancements to cyber-physical systems, analytical tools, and data interrogation tools which have transformed the consistency and robustness of biologics production through QbD (Wee et al., 2020, Biologicals, 63:53-61). A holistic and thorough understanding of the process is a vital component to designing a robust QbD roadmap which utilizes the necessary cyber-physical systems and analytical technologies for accurate real-time monitoring of a process's QTPP, CQAs and CPPs.

PAT can help to establish the critical control points (CPP) within a bioprocess. As biologic medicines are by their nature more complex, implementation of real-time process monitoring tools is more challenging compared to their small molecule counterparts. The increased complexity of biologics has meant that the analytical tools utilized in their manufacturing are significantly behind, and the space for new, more relevant technologies to conduct meaningful process monitoring is more necessary than ever before. Currently, there are various process sensors which are utilized to monitor the bioprocess. These sensors can be categorized by their monitoring location, either online, in-line, at-line or off-line. Online PAT measurements take place continuously; measurements are automatically taken as the monitor is in a sample loop attached to the vessel of interest (Gillespie et al., 2022, Biotechnology and Bioengineering, 119(2):423-434).

The continuous measurements using the online sensors enable the necessary conditions or substances to be added into the process, for example reagents or feed media. In-line process monitoring involves incorporating a probe directly into the vessel for direct sampling, thus enabling consistent and constant process monitoring. The advantage of in-line probes is that if the process continues to run, automatic sampling will be taken; this constant data acquisition permits deep process understanding as it can detect the exact moment a potential deviation occurred. The vibrational spectroscopic probes Raman and Fourier Transform Infra-Red are commonly utilized for real-time data acquisition in production bioreactors (De Beer et al., 2011, International Journal of Pharmaceutics, 417(1-2):32-47). At-line samples are conducted manually or automatically and processed by an external laboratory analyzing device situated near where the sample was drawn. Unfortunately, the process is not consistently monitored when using at-line measurements, and this can make it difficult to pinpoint the direct cause of the error if a deviation occurs.

Lastly, off-line measurements involve exporting a sample to an external laboratory piece of equipment for analysis. A major disadvantage is that the results from the sampling can often be time consuming, meaning that the measurement variant is largely disconnected from the bioprocess; this can result in several drawbacks, such as low temporal resolution and a restricted number of samples taken. Additionally, the potential of contamination to the product greatly increases, further contributing to its inaccuracy. For this reason, off-line measurements are not considered a true PAT tool. As biologic medicines continue to dominate the NME pipeline and pharmaceutical marketspace through their ground-breaking innovations and mechanisms of action to treat once untreatable diseases, much work is still required to further optimize and automate the bioprocess to improve production efficiency, consistency, quality and capabilities to enhance the safety of medicines.

Current cell culture analyzers typically rely on spectroscopic methods. A widely used spectroscopic off-line analyzer in the biopharmaceutical industry is the BioProfile FLEX, which encompasses three off-line analyzers, biochemical, cell counting, and vapor-pressure osmometer. It permits the analysis of 16 key cell culture components such as lactate, glutamine, glutamate, cell density/viability, and osmolarity for each sample (M&S Instruments., 2020). Unfortunately, the only analytical method that records the true state of the cells using this analytical machine is the cell density/viability; all other sensors only detect the nutrients and metabolites in the cell culture and are therefore not a true real-time representation of the cells. The BioProfile analyzer uses trypan blue dye to measure the VCD, followed by a visual inspection. The viable cells reject the dye if their membranes are intact, based on the theory that if the cell membrane is intact the cell is living and therefore the dye cannot permeate through the intact membrane (Chan et al., 2020, PLoS One, 15(1):0227950). Otherwise, the dye can permeate the cells, signifying a punctured or incomplete membrane. The cells appear dyed blue as the dye has colored the cytoplasm. Once the dye has interacted with the cells, high-resolution images are obtained by locating the cells with a fixed microscope objective using a high precision motion control system. Image processing can provide information about the cell count and viability, which are dependent on criteria such as viable/non-viable cells, average cell size and the brightness. Although samples of the bioprocess are analyzed each day using machines such as this bioanalyzer, they cannot be classified as real-time measurements.

Monitoring and controlling the viability and growth of the culture in real time is of critical importance, as it relays critical information about the progression of the culture in process controls and the overall evolution of the bioprocess. Within the upstream bioreactors are a series of probes monitoring pH, temperature, and dissolved oxygen. However, these probes cannot capture accurate real-time measurements of deviation events linked to the VCD. There is a distinct need in the field for in-situ cell culture analysers that can detect early signs of deviations to the culture and conduct accurate, real-time VCD measurements to a high degree of fidelity.

Disclosed herein are methods and systems for real-time monitoring of cell culture conditions, such as excursions or deviations relating to failure modes. The inventors have surprisingly discovered that permittivity measurements taken by real-time capacitance probes embedded in bioreactors can be used to derive impedance spectroscopy parameters that correlate to changes in bioreactor operating conditions, and therefore can be used for real-time monitoring of bioprocess deviations. In particular, changes over time in the critical frequency (f_{c}), permittivity increment (Δε), and alpha parameter (α) can be used to detect failure modes such as high temperature excursions, low dissolved oxygen, missed dextrose feeds and excess dextrose feeds. These, and other aspects of the invention are set forth in further detail below.

Research dedicated to dielectric spectroscopy is focused on investigating and quantifying a medium's dielectric property when exposed to an electric field. The electrical energy applied can either be stored or lost, depending on the properties of the medium (Markx and Davey, 1999, Enzyme and Microbial Technology, 25(3-5):161-171). Losing energy results in an exothermic reaction as the frictional motion of the charge escapes; this is referred to as resistive loss. If the charge is stored, it causes a charge separation and thus polarization of the medium's components (Carvell and Dowd, 2006, Cytotechnology, 50(1-3):35-48). In response to an applied electric field, both the medium's conductivity and permittivity are key factors, where the conductivity measures a medium's ability to conduct a charge and the permittivity is the polarizability and thus the ability of the medium to store a charge itself. Capacitance links these two phenomena together, as it is the quantity of charge withheld by a medium due the polarization of its constituents when subjected to an electric field (Markx and Davey). If a medium possesses a high permittivity, then it is polarized to a greater extent when an electric field is applied, thus storing more energy inside; this relationship exemplifies the link between capacitance and permittivity (Dabros et al., 2009, Bioprocess and Biosystems Engineering, 32:161-173). Permittivity is a unitless measurement, whereas the SI unit of capacitance is Farad (F).

Typically, both permittivity and conductivity remain as constant measurements when a specific electrical charge is applied to a medium; however, as the frequency increases, an inverse relationship between the two variables develops. An increase in the electric frequency will cause permittivity to decrease, thereby increasing the conductivity in a stepwise manner, called a dispersion. Dispersions, particularly at low frequencies, are prevalent in biological mediums due to the interfacial polarization events between the various materials that compose the cell. The three most prevalent dispersions identified in biological systems include α, β and λ, each representing a different polarization process within the cells (Dabros et al.).

Cells themselves can store electrical charge under the influence of an electric field, as illustrated by the single shell model (Surowei et al., 2023). The cell membrane, which remains intact in “healthy” cells, functions as an electrical insulator enveloping the predominantly ionic cytoplasm. Due to the insulating nature of the bilipid membrane, ions move in parallel to the applied current. At lower frequencies, the membrane surface reaches a state of complete polarization, allowing ample time for all ions to accumulate at the outer membrane and establish a charge separation (Bergin et al., 2022, Biotechnology Advances, 108048). The capacitive charge resulting from this phenomenon is quantified in picofarads (pF) and can be measured at one or several frequencies. It is essential to note that only viable cells with intact cellular membranes can be polarized and thus hold a capacitance charge. Information regarding the identity of the cell, such as the viable cell density and cell size, can be quantified from capacitance readings. When a cell is exposed to high frequencies, the membrane fails to act as an insulator to the electric field, and the resulting capacitance readings are negligible (Asami and Yonezawa, 1996, Biophysical Journal, 71(4):2192-2200).

The response of cells to an alternating current electric field can be characterized by distinct dispersion regions in their dielectric spectrum. The specific frequency applied to cells plays a crucial role in determining the system's characteristics for measurement. The α-dispersion region, which is predominantly linked with bacterial cells, occurs at frequencies below 0.1 MHz. Bacterial cells possess a significant negative surface charge due to their outer membrane composition, leading to the formation of a dipole with accumulated positive charges (Nasir et al., 2020, Journal of Engineering, 1-17). Thus, when exposed to a low-frequency field, these ions travel in a planar manner towards the cell surface. However, the impact of α-dispersion is minimal in mammalian cells and is typically disregarded in dielectric models. In the context of mammalian cells, the 3-dispersion region becomes particularly significant, when associated with charge accumulation at the surface of the bilipid membrane. Lastly, the γ-dispersion region focuses on measuring small molecules in aqueous solutions (Nasir et al.). However, for the purposes of this disclosure, the focus will be on the β-dispersion region within the frequency range of 0.1 to 50 MHz.

The β-dispersion region can be subdivided into distinct frequency regions of interest. Frequencies below 0.5 MHz provide information on the cell size by evaluating the relative volume of the cell in the fluid path. At this range, the membrane acts as a non-polarized barrier to the electric field. β relaxation is observed in the intermediate frequency range, which is up to approximately 6 MHz; this region is associated with calculating parameters related to the Cole-Cole plot, which is derived by superimposing the values from a range of frequencies (Bergin et al.). The relaxation occurs due to changes in the polarization of the plasma membrane as the electric frequency of the applied field increases. Above 6 MHz, a plateau is observed in the dispersion curve, although additional information can still be obtained. Systems capable of applying electric fields greater than 10 MHz can be used to investigate changes in cytoplasmic conductivity and determine the properties of intracellular components and particles.

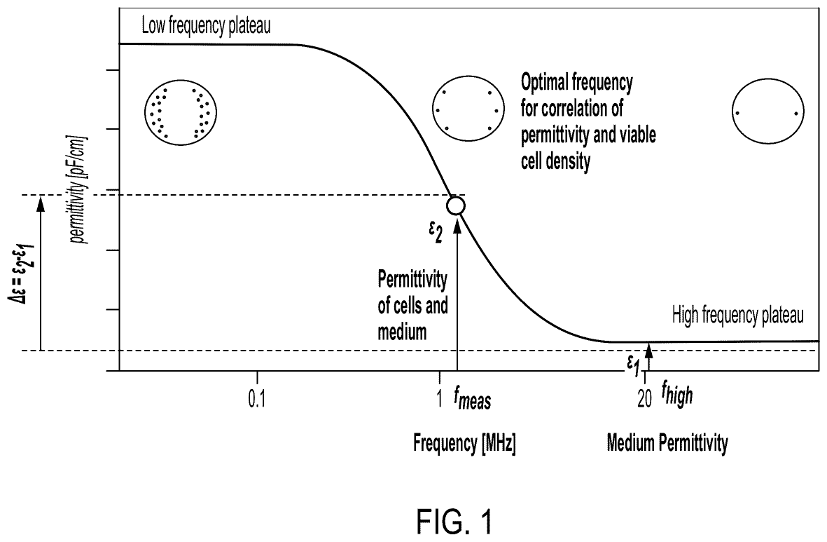

Cellular proliferation inside a bioreactor utilizing the principle of biocapacitance requires inline capacitance probes for its detection. These sterilizable probes are embedded with annular electrodes around the tip which emit a low-intensity electric field that enables viable cells with intact lipid bilayers to act as tiny capacitors holding a charge (Hamilton, 2021). Thus, increasing the cell growth directly increases the capacitance reading, and this is how biocapacitance can be effectively used as a real-time monitor of cellular growth. The precise frequency that is applied to the cells is subject to the cells present within the suspension; this is referred to as the measurement frequency. Mammalian cells' measurement frequency is 1 MHz when subject to frequency scanning, as depicted in FIG. 1. Capacitance probes deploy a dual measurement mode which monitors the lifecycle of the bioprocess, by deploying an increased frequency which is consistently corrected against the background permittivity measurement values, leaving the cellular suspension unaffected (Hamilton, 2021). Correlating permittivity to VCD is seen as an effective method of monitoring the progression of a bioprocess, with the link being most notable during the exponential growth phase (Bryan et al., 2014, Lab on a Chip, 14(3):569-576). As polarization is only possible at lower frequencies but not at higher frequencies, there is a persistent correction with the background permittivity measurements (Hamilton, 2021).

A commonly used model for the characterization of cell cultures using impedance spectroscopy is the Cole-Cole model. This model characterizes the dielectric spectroscopy relaxation phenomena using three parameters: the alpha parameter (α), critical frequency (f_{c}), and the permittivity increment (Δε) (Hanai et al., 1975, Bulletin of the Institutefor Chemical Research, Kyoto University, 58(5-6):534-547). These parameters are obtained by fitting the permittivity spectrum to the Cole-Cole model, where the frequency at which the rate of polarization is half complete is the f_{c}. Depending on the polarizability and the size of the cells, f_{c} changes. In biological suspensions it is unlikely that all cells present possess the same radii and internal conductance, which will cause distributions in the degree of relaxation. To overcome this discrepancy, the Cole-Cole model introduces the Cole-Cole parameter α which is utilised to factor in that the relaxations observed are broader than empirically calculated using the Debye equation, which assumes that all cells are the same size and is thus less accurate (Cole & Cole, 1941, The Journal of Chemical Physics, 9(4):341-351). This additional parameter more accurately portrays the dielectric dispersions present in a cellular suspension (Dabros et al.).

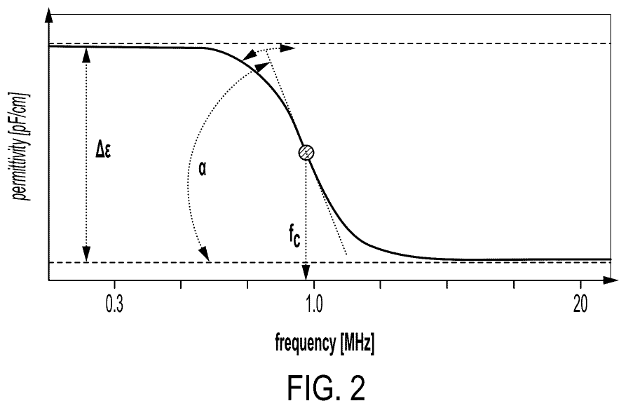

Because the degree of cellular polarization differs between high and low frequencies, multifrequency scanning across, for example, 17 frequencies provides a deeper understanding of the cellular physiology response throughout the bioprocess, as shown in FIG. 2 (Hamilton et al., 2021). The three empirically calculated parameters (α), (f_{c}) and (Δε) may be automatically fitted to the permittivity curve and displayed on the capacitance software interface. These parameters have been correlated with physical characteristics of the cell culture. F_{c} can provide information regarding the average cellular diameter: the greater the time spent in the bioprocess, the lower f_{c} (Bergin et al.). F_{c} when subject to a higher frequency range could indicate a reduction in cellular diameter. At low frequencies, Δε is a direct correlation to the VCD and a real-time representation of the maximum theoretical permittivity of the cellular suspension. The α parameter is indicative of the slope of the β-dispersion and can provide information regarding the distribution of the cell diameter within the culture; the greater the α value, the more homogenous the culture (Hamilton, 2021). Furthermore, this parameter is a representation of the steepness of the permittivity decline as frequency increases. Many cellular factors can cause alterations of the β-dispersion such as organelle content, cell size, intracellular conductivity, and membrane specific capacitance. Thus, relating β-dispersion to cellular morphology will show fluctuations throughout the bioprocess.

Over the past twenty years, capacitance technology has been one of the most reliable and advanced techniques for the on-line monitoring of cell culture biomass and the physiological state of cells. The technology is based on the mathematical model of Pauly and Schwan which describes the spherical cell's ability to act as capacitors under polarization at different frequencies (Petiot et al., 2017, Journal of Biotechnology, 242:19-29). The Cole-Cole model strengthens the capabilities of the original theory by incorporating dielectric constants such as Δε and f_{c}, which are linked to cellular biomass and the culture's physiological state, encapsulating membrane capacitance (Cm), cell biovolume (Bv), intracellular conductivity (σi), and cellular size (r). Both the f_{c} and Δε parameters are dependent on the biological and physical properties of the suspension (Bergin et al.). Biovolume evolution can be monitored using the Δε max signal, which is a function of cell size; as this increases, biovolume directly rises throughout the lifespan of the bioprocess.

Directly relating capacitance data to viable biomass is most physiologically relevant when there is a stable culture during the exponential phase of cell growth (Opel et al., 2010, Biotechnology Progress, 26(4):1187-1199). VCD is representative of the total live biovolume of the culture; however, it has been reported that in CHO cell cultures the correlation lacks accuracy as the cells enters the death phase where membrane permittivity, polarization and cell size are impacted. To overcome this discrepancy and increase the correlation between biovolume and biocapacitance, the viable cell volume (VCV) can be calculated, which is based on cellular size and quantity and is obtained using the following formula: VCV=4/3 πr³×VCD mm³/mL (Bergin et al.). The assumption when calculating VCV is that the cell culture possesses a spherical morphology, which becomes less accurate when present at low quantities or when the cells enter an apoptotic state.

Additionally, capacitance probes can measure conductivity (mS/cm), which directly relates to the degree of charged ions within a cell culture. A study by Caitlin Morris, et al., 2021 described the additional capability of capacitance probes for detecting contamination to a bioprocess by spiking a CHO cell culture with bacteria (Morris et al., 2021, Biotechnology Journal, 16(12):2100126). The study describes that an increase in the conductivity measurement was directly related to the contamination event, which displayed an upwards trajectory as the bacteria multiplied. Currently, bioreactor towers can monitor culture conditions such as pH and dissolved oxygen (DO), which are expected to be affected when contamination occurs. A major indicator of contamination to a bioprocess is a decline in DO and pH. This research demonstrated that although DO did decrease to 0% and pH declined, the conductivity profile displayed a sharp increase 32 hours before DO content or pH showed any signs of decline, indicating that capacitance probes can be used as an in-line early detection probe for monitoring failure modes, saving time and resources compared to the standard online measurements of pH and DO (Morris et al.). This conductivity perturbation within the process would not have been captured using standard online measurements of DO or through microscopic analysis of the samples. Conductivity spikes as seen here were reported in several studies when bacterial contamination and growth was monitored using capacitance probes.

In multiple reports, the maximum virus titre value was captured approximately 40 hours following the peak bio-capacitance; these studies displayed that the bioreactors containing capacitance probes contributed an increment of three orders of magnitude in the total virus concentration in comparison to the vessels housing no capacitance probes (Gränicher et al., 2021, Biotechnology and Bioengineering, 118(12):4720-4734). The critical frequency (f_{c}) based on the frequency scan could correlate the scan to the influenza virus production kinetics, demonstrating how bio-capacitance probes can be used to monitor the critical phases of culture production (Petiot et al.).

The aims of the experiments disclosed herein included assessing the capabilities of capacitance probes as an early indicator at detecting a series of failure modes of particular interest in cell culture using the dielectric spectroscopy relaxation phenomena parameters α, f_{c} and Δε. These parameters are obtained by fitting the permittivity spectrum to the Cole-Cole model, where the frequency at which the rate of polarization is half complete is the f_{c}. These empirically calculated parameters obtained through a frequency scan using the capacitance probes were correlated to several cell culture outputs, such as alterations in cell density, cell size, and cellular distribution. The potential capability of capacitance probes as an early indicator of deviations to a process can augment the biomanufacturing investigational toolkit.

The experiments disclosed herein involved the investigation of specific failure modes in the production of an exemplary molecule. The cell growth was monitored throughout seed train (ST) and production unit operations using both capacitance probes and a bioanalyzer. The potential for capacitance probes to determine early decisions when exposed to specific failure modes was assessed throughout seed train and into production. A goal of the study was to test the capabilities of capacitance probes as an early detection PAT for specific failure modes in cell culture bioprocessing, in turn providing enhanced cell culture control opportunities by reducing time, lowering operational costs, and increasing reproducibility.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, bispecific antibodies, and antigen-binding proteins.

Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated by reference). In some aspects, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

As used herein, the term “recombinant protein” refers to a protein produced as the result of the transcription and translation of a gene carried on a recombinant expression vector that has been introduced into a suitable host cell. In certain aspects, the recombinant protein can be an antibody, for example, a chimeric, humanized, or fully human antibody. In certain aspects, the recombinant protein can be an antibody of an isotype selected from group consisting of: IgG, IgM, IgA1, IgA2, IgD, or IgE. In certain aspects the antibody molecule is a full-length antibody (e.g., an IgG1) or alternatively the antibody can be a fragment (e.g., an Fc fragment or a Fab fragment).

As used herein, the term “recombinant host cell” (or simply “host cell”) includes a cell into which a recombinant expression vector coding for a protein of interest has been introduced. It should be understood that such a term is intended to refer not only to a particular subject cell but to a progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the kingdoms of life. In one aspect, eukaryotic cells include protist, fungal, plant and animal cells. In a further aspect, host cells include eukaryotic cells such as plant and/or animal cells. The cells can be mammalian cells, fish cells, insect cells, amphibian cells or avian cells. In a particular aspect, the host cell is a mammalian cell. A wide variety of mammalian cell lines suitable for growth in culture are available from the American Type Culture Collection (Manassas, Va.) and other depositories as well as commercial vendors. Cells that can be used in the processes of the invention include, but are not limited to, MK2.7 cells, PER-C6 cells, Chinese hamster ovary cells (CHO), such as CHO-K1 (ATCC CCL-61), (Chasin et al., 1986, Som. Cell Molec. Genet., 12:555-556; Kolkekar et al., 1997, Biochemistry, 36: 10901-10909; and WO 01/92337 A2), dihydrofolate reductase negative CHO cells (CHO/-DHFR, Urlaub and Chasin, 1980, Proc. Natl. Acad. Sci. USA, 77:4216), and dp12.CHO cells (U.S. Pat. No. 5,721,121); monkey kidney cells (CV1, ATCC CCL-70); monkey kidney CV1 cells transformed by SV40 (COS cells, COS-7, ATCC CRL-1651); HEK293 cells, Sp2/0 cells, 5L8 hybridoma cells, Daudi cells, EL4 cells, HeLa cells, HL-60 cells, K562 cells, Jurkat cells, THP-1 cells, Sp2/0 cells, primary epithelial cells (e.g., keratinocytes, cervical epithelial cells, bronchial epithelial cells, tracheal epithelial cells, kidney epithelial cells and retinal epithelial cells) and established cell lines and their strains (e.g., human embryonic kidney cells (e.g., HEK293 cells, or HEK293 cells subcloned for growth in suspension culture, Graham et al., 1977, J. Gen. Virol., 36:59); baby hamster kidney cells (BHK, ATCC CCL-10); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982, Annals NY Acad. Sci., 383:44-68); MCR 5 cells; FS4 cells; PER-C6 retinal cells, MDBK (NBL-1) cells, 911 cells, CRFK cells, MDCK cells, BeWo cells, Chang cells, Detroit 562 cells, HeLa 229 cells, HeLa S3 cells, Hep-2 cells, KB cells, LS 180 cells, LS 174T cells, NCI-H-548 cells, RPMI 2650 cells, SW-13 cells, T24 cells, WI-28 VA13, 2RA cells, WISH cells, BS-C-I cells, LLC-MK₂ cells, Clone M-3 cells, 1-10 cells, RAG cells, TCMK-1 cells, Y-1 cells, LLC-PK₁ cells, PK(15) cells, GH₁ cells, GH₃ cells, L2 cells, LLC-RC 256 cells, MH₁C₁ cells, XC cells, MDOK cells, VSW cells, and TH-I, B1 cells, or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, MiCl₁ cells, CV-1 cells, COS-1 cells, COS-3 cells, COS-7 cells, African green monkey kidney cells (VERO-76, ATCC CRL-1587; VERO, ATCC CCL-81); DBS-FrhL-2 cells, BALB/3T3 cells, F9 cells, SV-T2 cells, M-MSV-BALB/3T3 cells, K-BALB cells, BLO-11 cells, NOR-10 cells, C₃H/IOTI/2 cells, HSDM₁C₃ cells, KLN205 cells, McCoy cells, Mouse L cells, Strain 2071 (Mouse L) cells, L-M strain (Mouse L) cells, L-MTK (Mouse L) cells, NCTC clones 2472 and 2555, SCC-PSA1 cells, Swiss/3T3 cells, Indian muntac cells, SIRC cells, Cr cells, and Jensen cells, or derivatives thereof) or any other cell type known to one skilled in the art.

In some aspects, cells used in the invention are CHO cells, HEK293 cells, BHK cells, or derivatives thereof. In some aspects, the cells express a protein of interest, such as a biotherapeutic protein. In some aspects, the cell used in the production of the protein is a mammalian cell capable of producing a biotherapeutic, such as CHO, HEK293, and BHK cell, or any derivatives thereof. In one embodiment, the cell is a CHO cell, such as a CHO-K1 cell. ANTIBODIES

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In some aspects, the protein of interest is a human antibody. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

Interleukin-4 (IL-4) and interleukin-13 (IL-13) are key cytokines in driving allergic and T helper cell type 2 (Th2) polarized inflammatory processes. IL-4 and IL-13 signaling is mediated through heterodimeric receptor complexes, in which IL-4 receptor alpha (IL-4Rα) is a shared receptor subunit for both IL-4 and IL-13 signaling. Thus, IL-4Rα is an attractive therapeutic target because it provides a single target for blocking both IL-4 and IL-13 signaling. In some aspects, the protein of interest is an anti-IL-4R antibody, or an antigen-binding fragment thereof. Antibodies to hIL-4Rα are described in, for example, U.S. Pat. Nos. 5,717,072, 7,186,809 and 7,605,237.

In some aspects, the anti-IL-4R antibody is a human IgG antibody. In various aspects, the anti-IL-4R antibody is a human antibody of isotype IgG1, IgG2, IgG3 or IgG4, or mixed isotype. In some aspects, the anti-IL-4R antibody is a human IgG1 antibody. In some aspects, the anti-IL-4R antibody is a human IgG4 antibody. In any of the aspects discussed above or herein, the anti-IL-4R antibody may comprise a human kappa light chain. In any of the aspects discussed above or herein, the anti-IL-4R antibody may comprise a human lambda light chain.

The antibodies of the disclosure may, in some aspects, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain aspects, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some aspects, an antibody fragment comprises a sufficient amino acid sequence of the parent antibody of which it is a fragment that it binds to the same antigen as does the parent antibody; in some aspects, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ,-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

An “isolated antibody,” as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Rα).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4Rα molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material and/or chemicals. However, in some instances, the isolated antibody may be copurified with a phospholipase expressed by a mammalian cell line (e.g., CHO cells) from which the anti-IL-4R antibody is produced.

For example, for antibody production, aspects of the inventions are amenable for research and production use for diagnostics and therapeutics based on all major antibody classes, namely IgG, IgA, IgM, IgD, and IgE. IgG is a preferred class, and includes subclasses IgG1 (including IgG1λ and IgG1κ), IgG2, IgG3, and IgG4. In some aspects, the protein of interest or polypeptide of interest is an antibody, a human antibody, a humanized antibody, a chimeric antibody, a monoclonal antibody, a multispecific antibody, a bispecific antibody, an antibody fragment, an antigen-binding antibody fragment, a single chain antibody, a diabody, triabody or tetrabody, a Fab fragment or a F(ab′)2 fragment, an IgD antibody, an IgE antibody, an IgM antibody, an IgG antibody, an IgG1 antibody, an IgG2 antibody, an IgG3 antibody, an IgG4 antibody, a fusion protein, a receptor fusion protein, an antibody-derived protein, or combinations thereof. In one aspect, the antibody is an IgG1 antibody. In one aspect, the antibody is an IgG2 antibody. In one aspect, the antibody is an IgG4 antibody. In one aspect, the antibody is a chimeric IgG2/IgG4 antibody. In one aspect, the antibody is a chimeric IgG2/IgG1 antibody. In one aspect, the antibody is a chimeric IgG2/IgG1/IgG4 antibody. Derivatives, components, domains, chains, and fragments of the above are also included.

In some aspects, the antibody is selected from the group consisting of an anti-Programmed Cell Death 1 antibody (e.g. an anti-PD1 antibody as described in U.S. Pat. App. Pub. No. US2015/0203579A1), an anti-Programmed Cell Death Ligand-1 antibody (e.g. an anti-PD-L1 antibody as described in in U.S. Pat. App. Pub. No. US2015/0203580A1), an anti-D114 antibody, an anti-Angiopoietin-2 antibody (e.g. an anti-ANG2 antibody as described in U.S. Pat. No. 9,402,898), an anti-Angiopoietin-Like 3 antibody (e.g. an anti-AngPtl3 antibody as described in U.S. Pat. No. 9,018,356), an anti-platelet derived growth factor receptor antibody (e.g. an anti-PDGFR antibody as described in U.S. Pat. No. 9,265,827), an anti-Erb3 antibody, an anti-Prolactin Receptor antibody (e.g. anti-PRLR antibody as described in U.S. Pat. No. 9,302,015), an anti-Complement 5 antibody (e.g. an anti-C5 antibody as described in U.S. Pat. App. Pub. No US2015/0313194A1), an anti-TNF antibody, an anti-epidermal growth factor receptor antibody (e.g. an anti-EGFR antibody as described in U.S. Pat. No. 9,132,192 or an anti-EGFRvIII antibody as described in U.S. Pat. App. Pub. No. US2015/0259423A1), an anti-Proprotein Convertase Subtilisin Kexin-9 antibody (e.g. an anti-PCSK9 antibody as described in U.S. Pat. No. 8,062,640 or U.S. Pat. App. Pub. No. US2014/0044730A1), an anti-Growth And Differentiation Factor-8 antibody (e.g. an anti-GDF8 antibody, also known as anti-myostatin antibody, as described in U.S. Pat. No. 8,871,209 or U.S. Pat. No. 9,260,515), an anti-Glucagon Receptor (e.g. anti-GCGR antibody as described in U.S. Pat. App. Pub. Nos. US2015/0337045A1 or US2016/0075778A1), an anti-VEGF antibody, an anti-IL1R antibody, an interleukin 4 receptor antibody (e.g., an anti-IL4R antibody as described in U.S. Pat. App. Pub. No. US2014/0271681A1 or U.S. Pat. No. 8,735,095 or U.S. Pat. No. 8,945,559), an anti-interleukin 6 receptor antibody (e.g. an anti-IL6R antibody as described in U.S. Pat. Nos. 7,582,298, 8,043,617 or 9,173,880), an anti-IL1 antibody, an anti-IL2 antibody, an anti-IL3 antibody, an anti-IL4 antibody, an anti-IL5 antibody, an anti-IL6 antibody, an anti-IL7 antibody, an anti-interleukin 33 (e.g. anti-IL33 antibody as described in U.S. Pat. App. Pub. Nos. US2014/0271658A1 or US2014/0271642A1), an anti-Cluster of differentiation 3 antibody (e.g. an anti-CD3 antibody, as described in U.S. Pat. App. Pub. Nos. US2014/0088295A1 and US20150266966A1, and in U.S. Application No. 62/222,605), an anti-Cluster of differentiation 20 antibody (e.g. an anti-CD20 antibody as described in U.S. Pat. App. Pub. Nos. US2014/0088295A1 and US20150266966A1, and in U.S. Pat. No. 7,879,984), an anti-CD19 antibody, an anti-CD28 antibody, an anti-Cluster of Differentiation-48 antibody (e.g. anti-CD48 antibody as described in U.S. Pat. No. 9,228,014), an anti-Fel d1 antibody (e.g. as described in U.S. Pat. No. 9,079,948), an anti-influenza virus antibody, an anti-Respiratory syncytial virus antibody (e.g. anti-RSV antibody as described in U.S. Pat. App. Pub. No. US2014/0271653A1), an anti-Middle East Respiratory Syndrome virus antibody (e.g. an anti-MERS-CoV antibody as described in U.S. Pat. App. Pub. No. US2015/0337029A1), an anti-Ebola virus antibody (e.g. as described in U.S. Pat. App. Pub. No. US2016/0215040), an anti-Zika virus antibody, an anti-Severe Acute Respiratory Syndrome (SARS) antibody (e.g., an anti-SARS-CoV antibody), an anti-COVID-19 antibody (e.g., an anti-SARS-CoV-2 antibody), an anti-Lymphocyte Activation Gene 3 antibody (e.g. an anti-LAG3 antibody, or an anti-CD223 antibody), an anti-Nerve Growth Factor antibody (e.g. an anti-NGF antibody as described in U.S. Pat. App. Pub. No. US2016/0017029 and U.S. Pat. Nos. 8,309,088 and 9,353,176) and an anti-Activin A antibody. In some aspects, the bispecific antibody is selected from the group consisting of an anti-CD3×anti-CD20 bispecific antibody (as described in U.S. Pat. App. Pub. Nos. US2014/0088295A1 and US20150266966A1), an anti-CD3×anti-Mucin 16 bispecific antibody (e.g., an anti-CD3×anti-Muc16 bispecific antibody), an anti-CD3×BCMA bispecific antibody, and an anti-CD3×anti-Prostate-specific membrane antigen bispecific antibody (e.g., an anti-CD3×anti-PSMA bispecific antibody). See also U.S. Patent Publication No. US 2019/0285580 A1. Also included are a Met×Met antibody, an agonist antibody to NPR1, an LEPR agonist antibody, a MUC16×CD28 antibody, a GITR antibody, an IL-2Rg antibody, an EGFR×CD28 antibody, a Factor XI antibody, antibodies against SARS-CoV-2 variants, a Fel d 1 multi-antibody therapy, and a Bet v 1 multi-antibody therapy. Derivatives, components, domains, chains and fragments of the above also are included. In one aspect, the protein of interest or polypeptide of interest comprises a combination of any of the foregoing.

Cells that produce exemplary antibodies can be cultured according to the inventions. In some aspects, the protein of interest or polypeptide of interest is selected from the group consisting of Alirocumab, Atoltivimab, Maftivimab, Odesivimab, Odesivimab-ebgn, Casirivimab, Imdevimab, Cemplimab and Cemplimab-rwlc (human IgG4 monoclonal antibody that binds to PD-1), Sarilumab, Fasinumab, Nesvacumab, Dupilumab (human monoclonal antibody of the IgG4 subclass that binds to the IL-4R alpha (α) subunit and thereby inhibits Interleukin 4 (IL-4) and Interleukin 13 (IL-13) signaling), Trevogrumab, Evinacumab, Evinacumab-dgnb, Fianlimab, Garetosmab, Itepekimab, Odrononextamab, Pozelimab, Rinucumab, and modifications, truncations, and variations thereof. In a specific aspect, the cells cultured according to the invention are CHO-K1 cells and the protein of interest expressed by the cells is Dupilumab.

Additional exemplary antibodies include Ravulizumab-cwvz, Abciximab, Adalimumab, Adalimumab-atto, Ado-trastuzumab, Alemtuzumab, Atezolizumab, Avelumab, Basiliximab, Belimumab, Benralizumab, Bevacizumab, Bezlotoxumab, Blinatumomab, Brentuximab vedotin, Brodalumab, Canakinumab, Capromab pendetide, Certolizumab pegol, Cetuximab, Denosumab, Dinutuximab, Durvalumab, Eculizumab, Elotuzumab, Emicizumab-kxwh, Emtansine alirocumab, Evolocumab, Golimumab, Guselkumab, Ibritumomab tiuxetan, Idarucizumab, Infliximab, Infliximab-abda, Infliximab-dyyb, Ipilimumab, Ixekizumab, Mepolizumab, Necitumumab, Nivolumab, Obiltoxaximab, Obinutuzumab, Ocrelizumab, Ofatumumab, Olaratumab, Omalizumab, Panitumumab, Pembrolizumab, Pertuzumab, Ramucirumab, Ranibizumab, Raxibacumab, Reslizumab, Rinucumab, Rituximab, Secukinumab, Siltuximab, Tocilizumab, Trastuzumab, Ustekinumab, and Vedolizumab.

In addition to next generation products, the inventions also are applicable to production of biosimilars. Biosimilars are defined in various ways depending on the jurisdiction, but share a common feature of comparison to a previously approved biological product in that jurisdiction, usually referred to as a “reference product.” According to the World Health Organization, a biosimilar is a biotherapeutic product similar to an already licensed reference biotherapeutic product in terms of quality, safety and efficacy, and is followed in many countries, such as the Philippines.

A biosimilar in the U.S. is currently described as (A) a biological product that is highly similar to the reference product notwithstanding minor differences in clinically inactive components; and (B) there are no clinically meaningful differences between the biological product and the reference product in terms of the safety, purity, and potency of the product. In the U.S., an interchangeable biosimilar or product may be substituted for the previous product without the intervention of the health care provider who prescribed the previous product. In the European Union, a biosimilar is a biological medicine highly similar to another biological medicine already approved in the EU (called “reference medicine”) and includes consideration of structure, biological activity, efficacy, and safety, among other things, and these guidelines also are followed by Russia. In China, a biosimilar product currently refers to biologics that contain active substances similar to the original biologic drug and is similar to the original drug in terms of quality, safety, and effectiveness, with no clinically significant differences. In Japan, a biosimilar currently is a product that has bioequivalent/quality-equivalent quality, safety, and efficacy to a reference product already approved in Japan. In India, biosimilars currently are referred to as “similar biologics,” and refer to a similar biologic product which is similar in terms of quality, safety, and efficacy to an approved reference biological product based on comparability. In Australia, a biosimilar medicine currently is a highly similar version of a reference biological medicine. In Mexico, Columbia, and Brazil, a biosimilar currently is a biotherapeutic product that is similar in terms of quality, safety, and efficacy to an already licensed reference product. In Argentina, a biosimilar currently is derived from an original product (a comparator) with which it has common features. In Singapore, a biosimilar currently is a biological therapeutic product that is similar to an existing biological product registered in Singapore in terms of physicochemical characteristics, biological activity, safety and efficacy. In Malaysia, a biosimilar currently is a new biological medicinal product developed to be similar in terms of quality, safety and efficacy to an already registered, well established medicinal product. In Canada, a biosimilar currently is a biologic drug that is highly similar to a biologic drug that was already authorized for sale. In South Africa, a biosimilar currently is a biological medicine developed to be similar to a biological medicine already approved for human use. Production of biosimilars and its synonyms under these and any revised definitions can be undertaken according to the inventions.

### Pharmaceutical Formulations

The terms “composition,” “formulation,” and “formulated drug substance” (FDS) as used in the present disclosure refer to a combination of two or more pharmaceutical ingredients for inclusion in a drug product. A composition, formulation, or FDS may be, for example, a liquid composition including an active pharmaceutical ingredient, such as an antibody, and an excipient, such as a stabilizer or surfactant. A composition, formulation, or FDS may include multiple excipients. A composition, formulation, or FDS may also include other constituents, such as host cell proteins co-purified with a protein of interest.

The term “drug product” (DP) as used in the present disclosure refers to a dosage form comprising an amount of a FDS for packaging, shipment, or administration. For example, a drug product may be a pre-filled syringe holding a volume of FDS for administration to a patient.

As used herein, a “protein pharmaceutical product,” “biopharmaceutical product,” “pharmaceutical formulation,” “pharmaceutical composition,” or “biotherapeutic” includes an active ingredient which can be fully or partially biological in nature. In one aspect, the protein pharmaceutical product can comprise a peptide, a protein, a fusion protein, an antibody, an antigen, vaccine, a peptide-drug conjugate, an antibody-drug conjugate, a protein-drug conjugate, cells, tissues, or combinations thereof. In another aspect, the protein pharmaceutical product can comprise a recombinant, engineered, modified, mutated, or truncated version of a peptide, a protein, a fusion protein, an antibody, an antigen, vaccine, a peptide-drug conjugate, an antibody-drug conjugate, a protein-drug conjugate, cells, tissues, or combinations thereof.

The pharmaceutical formulations of the present disclosure may, in certain aspects, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

Experimental protocol. A purpose of the working examples set forth below was to assess the ability of capacitance probes embedded within four bioreactors to continuously detect viable cell density (VCD), as well as a series of specific failure modes inflicted on the cell culture. Four distinct failure modes were assessed, one in the seed train (ST) and three in the production phase of the bioprocess. One cell line was utilized throughout the investigation, comprising recombinant CHO cells producing Dupilumab, cultured in a chemically defined medium.

Cell line and media. The cell line cultured throughout the study required chemically defined medium with a defined composition throughout the experiment. Two distinct media were used throughout the duration of the experiment. One was a ST medium and the other was a separate chemically defined production medium; each was selected to meet the specific requirements of the cells during the bioprocess. Additionally, the cells required nutrient feeds at specified days within the production phase, which promoted their growth and production of the target protein.

Media preparation. All raw material component lots of medium were prepared according to a prespecified batch sheet to align with the nutrient requirements of the cells in the large-scale bioprocess. Each media component was carefully weighed on an electronic balance and individually added to a specific quantity of warmed water. The components were constantly mixed using a magnetic stir bar until the contents became transparent. All media was stored inside a refrigerated unit and given a required expiry per the specific recipe.

On predefined days of the bioprocess in production, at specific times, a set volume of nutrient feed was prepared. Nutrient feed had to be prepared the day it was required to ensure that it did not fall outside the window of expiry, which was 7 hours after the first component was added. Much like the nutrient media, each component was weighed on an electronic balance and added individually to warmed water where it was stirred using a magnetic stir bar. Furthermore, two additional nutrient preparations were used to ensure cellular proliferation on certain days of the study. The timing of each of these feeds was in accordance with that of an in-house large-scale manufacturing floor to ensure that the model was representative of the actual drug production.

Seed Culture. Once transferred from a wave bioreactor 1 and inoculated into four N-3 bioreactors, the VCD was assessed using a combination of a bioanalyzer and capacitance probes. Capacitance probes were embedded within four bioreactors across N-3, N-2 and N-1.

Vial Thaw. Vial thaw was the first step in the seed train process. Vial thaw included carefully thawing a vial of frozen cells inside a water bath over a set number of minutes using a figure eight motion to ensure that thawing continued evenly. The thawed cells were inoculated into a shake-flask with the necessary quantity of warmed ST media. The shake flask was placed inside an incubator with set CO₂ and temperature setpoints, specific for the thawed cell line. The shaking base was switched on to ensure that the cells were subjected to the media nutrients continuously over the process. Several predefined sampling times prompted samples to be taken from the shake flask, where they were run on the bioanalyzer to ensure the culture was progressing and meeting all the in-process controls (IPCs).

Seed Expansion in Wave Bioreactor 1. The necessary quantity of ST media was aliquoted into a media bag using a welder and peristaltic pump and placed into an incubator to warm, so as not to shock the cells. The warmed media was then transferred into an inflated wave bioreactor 1. The wave bioreactor was fixed into the rocking unit and all the setpoints for the cell line (outlined in the manufacturing records (MR)) were input onto the digital interface of the system. Subsequently, the cells inside the shake-flask were transferred into the wave bioreactor 1. Post-inoculation, the cells were sampled using the bioanalyzer. Additionally, at set sampling times, samples were taken to assess the progression of the cell culture. In accordance with the MR, once the cell expansion time was met, a set volume of additional pre-warmed media was added into the wave bioreactor 1. A post-media addition sample was then taken and analyzed using the bioanalyzer.

Seed Expansion in Wave Bioreactor 2. The necessary volume of ST media was aliquoted using a welder and peristaltic pump and allowed to warm inside an incubator prior to transferring into an inflated wave bioreactor 2. The wave bioreactor 2 was fit into the rocking platform and all the necessary setpoints for the cell line were input onto the digital interface of the system in accordance with the MR. Once the expansion times were all met for the cell culture inside the rocking wave bioreactor 1 and a sample was taken and analyzed using the bioanalyzer confirming this, the culture was transferred into the wave bioreactor 2. After inoculation, a sample of cell culture was taken from the wave bioreactor 2 and analyzed using the bioanalyzer. Once the expansion time for the cell culture was met, samples were taken and analyzed using the bioanalyzer. A specific volume of ST media was aliquoted and warmed in an incubator prior to adding to the wave bioreactor 2. Another sample was taken post-media addition and analyzed using the bioanalyzer.

Seed Expansion in the Bioreactors—Preparation, Inoculation, Sampling and Excursion. Prior to autoclaving, each pH and DO probe for the four bioreactors was calibrated. The pH probes were calibrated by placing the probes into a series of standard solutions, one slightly acidic and the other neutral. The DO probes were 0% calibrated by sparging nitrogen, creating an oxygen-free environment. Four bioreactors were washed in a high strength cleaning solution, assembled and autoclaved. The required volume of ST calibration media was aliquoted into media bags and stored in a refrigerated unit. Prior to addition to the autoclaved bioreactors the media was prewarmed inside an incubator for a set time period in accordance with the MR. Once the media was transferred into each bioreactor, the setpoint parameters were input into the bioreactor control tower and the appropriate parameters switched on. The DO probes were calibrated to 100% oxygen saturation. Specific volumes of three necessary solutions were aliquoted into transfer bottles and welded onto each bioreactor. The lines for each of these solutions was subsequently primed. The appropriate volumes of ST media were aliquoted into media bags and prewarmed inside an incubator for a predefined time period as per the MR. The calibration media for each bioreactor was drained and the pre-warmed media was transferred into each bioreactor. A specific volume of cells was transferred from wave bioreactor 2 into a media bag and from that media bag the cells were split into four set volumes in media bags. These bags were welded onto the bioreactor towers and transferred into the N-3 bioreactors. All necessary control parameters were switched on. Once the cells were added to each bioreactor, the capacitance probe Toughbook software was marked as “inoc”, signifying that inoculation had occurred. Post-inoculation, once all parameters on the bioreactor control tower had stabilized, a sample of each bioreactor was taken and analyzed on the bioanalyzer. The VCD and % viability was assessed to ensure that the cells were within the IPC ranges. On day 1 of N-3, the first excursion was inflicted to bioreactor A3, where a five degree temperature increase was performed. The whole excursion lasted forty minutes and encapsulated a ten-minute temperature increase, the high temperature held for twenty minutes, and lastly a ten-minute temperature decreases where ice packs were held to the sides of the vessel to ensure rapid cooling took place. Transfers to N-2 and N-1 occurred when the expansion times were met and the VCD and % viability were within the IPCs. Each bioreactor was sampled once a day and pH adjustments were performed when required.

Production Bioreactor—Preparation, Inoculation, Sampling and Excursions. The predefined production bioreactor settings were input on the bioreactor control tower for each vessel as per the MR. The required quantities of production media were aliquoted into media bags and placed into an incubator to warm for a predefined time range as outlined by the MR. Following meeting all the expansion times in N-1, a final sample was taken from each bioreactor and analyzed using the bioanalyzer to ensure that all the IPCs were met for VCD and % Viability. Once the IPCs were met, the contents of each vessel were drained and the prewarmed production media was added to the bioreactors. A specific quantity of the drained cell culture was aliquoted into media bags and welded onto each of the four bioreactors. After inoculation into the N phase, a sample of the culture was taken and analyzed using the bioanalyzer. Each day of the production phase of the bioprocess, a sample was taken of the cell culture and analyzed using the bioanalyzer. When the bioprocess progressed to a particular day of the production phase, two daily samples were taken and analyzed using the bioanalyzer. After each sample, pH adjustments were performed, and glucose additions were added as required based on the values obtained via the MR. Throughout the N phase, certain prespecified days of the process required an additional nutrient feed by adding a specific combination of four solutions, depending on the day. One of these solutions could only be made the day it was required, as it had a short window of expiry as per the MR. Over a 24 hour period during the middle stage of the production phase, three additional excursions were inflicted onto the cells. A3 was subject to a low dissolved oxygen (DO) excursion, where the DO was a fifth of the original value. A5 endured two missed dextrose feeds, and A6 was given a double dextrose feed at one sampling point during that period. All sampling times and DO values were reset to the original setpoints once the 24 hour excursion period elapsed. Once all the necessary expansion times were met during the production phase, a final sample of cell culture from each bioreactor was taken and analyzed using the bioanalyzer. No harvest process was carried out for this study, and thus all controls on the bioreactor control tower were switched off for each tower and the culture drained and disposed of. Subsequently, all bioreactors were cleaned with a high strength cleaning solution and all probes stored correctly.

Offline Analytics. All the nutrients, metabolites, VCD and viability of the cell culture throughout the ST and production phases were analyzed using the bioanalyzer. When taking each of the samples, an initial syringe was pulled to clear the sampling line and was discarded prior to the actual sample being taken and subsequently analyzed using the bioanalyzer. When analyzing the sample using the bioanalyzer, the culture was aspirated while the needle of the machine processed the culture. When the results of the sample were displayed on the digital interface of the bioanalyzer, they were recorded. It was of major importance that the correct temperature, cell dilution ratio and nutrient/metabolite dilution ratio were selected, as the VCD and pH of the sample were dependent on these values being correct. For each sample taken, the sampling time was recorded.

Online Capacitance Measurements. The capacitance software continuously detected a series of capacitance measurements such as permittivity, conductivity, the characteristic frequency, alpha parameter, and the permittivity increment across seventeen different frequencies at set time points throughout ST and production. Only when an excursion was occurring did the time interval between data acquisition change, where excursion time points retrieved data more frequently. The capacitance probe was connected to an external toughbook laptop using a VP8 connector. The laptop housed the capacitance software and digital interface where the data acquisition was displayed on the screen as it was continuously detected at the specific time points. Periodically, data was exported from the software to a location on the toughbook laptop.

Software Management. Prior to inoculating each bioreactor, the capacitance software was set up with a specific experiment for each bioreactor. This involved naming the experiment and outlining how frequently the sampling time points should occur. The experiment was allowed to run by clicking the “Start Experiment” button prior to transferring cells so that each bioreactor just contained the required volume of prewarmed media. Just before draining in the cell culture the “Mark Zero” button was pressed, and once all cells were added to the bioreactor the “Inoculation” button was selected. When carrying out an excursion, the previous experiment was stopped by selecting the “Stop Experiment” button. A new experiment was then created with a shorter time interval between sampling points; for the N-3 excursion a three second interval was selected, and for the N excursions a thirty second interval was selected. After the time period of the excursion elapsed, the experiment was stopped and another new one was started with the original data acquisition time points. The nomenclature for the experiments indicated what was occurring within each experiment, for example “A3 Pre-Excursion”, “A3 During__Excursion” and “A3 Post Excursion”. After each experiment was ceased the data was exported.

Data Analysis and Handling. The data acquired via the bioanalyzer was trended for all nutrients, metabolites, VCD and viability to ensure that the bioprocess transpired in accordance with historic trends. Additionally, the data acquired using the online capacitance probes was trended and compared and correlated with the offline measurements. Using the capacitance data (permittivity) from the online measurements in combination with the off-line (VCD) measurements enabled the real-time VCD to be derived using a specific equation created from this correlation. Additionally, the three model parameters f_{c}, α and Δε were all trended with the permittivity to assess if they could act as an early detection device for monitoring specific failure modes to the bioprocess. From combining the data acquired from the online and off-line measurements, additional information about the cell culture was investigated, for example the viable cell volume (VCV). All the data was analyzed using online software. To correctly analyze the online capacitance data, all the separate experimental files from the toughbook laptop were combined prior to data analysis.

### Example 1. Establishment of a Linear Correlation Model

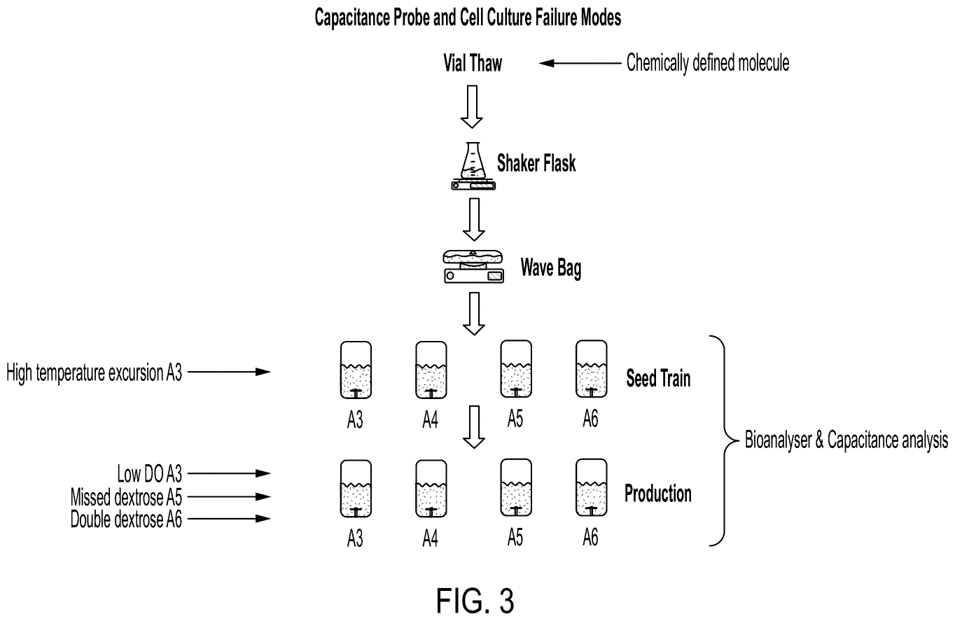

A Dupilumab-producing CHO cell line was used to carry out the following investigation using four bioreactors in the seed train (ST) and production phases of the bioprocess, as shown in FIG. 3. Each bioreactor was autoclaved with a capacitance probe, pH probe and DO probe. The primary excursion took place in the ST phase using one bioreactor (A3); this excursion subjected the culture to an elevated temperature by five degrees celsius. Three additional excursions took place in the production phase of the process, where the cells endured a missed dextrose addition (A5), double dextrose addition (A6), and low DO (A3), respectively. Bioreactor A6 was not provided with any dextrose, contrary to the dextrose addition value outlined via the relevant MR. The cell culture in A6 was given double the quantity of the dextrose value outlined in the relevant MR. Lastly, A3 was only subjected to 20% of the necessary DO quantity outlined in the relevant MR. A4 acted as the control in these experiments and thus was not subjected to any of the indicated failure mode excursions. Throughout the duration of the study, samples were physically taken and analyzed on the bioanalyzer while the capacitance probes continuously monitored the bioprocess throughout. The terminology N-5, N-4, N-3, N-2, and N-1 in this disclosure refers to the phases of the ST bioprocess, while N refers to the production phase.

Capacitance or permittivity readings were obtained by culturing cells in a scaled down model of a large-scale industrial bioprocess. Capacitance probes were added into four bench top bioreactors and standard cellular cultivations were carried out throughout ST and into the production phase. Periodic offline samples of the cell culture from each bioreactor were obtained and analyzed using a bioanalyzer, which utilized trypan blue dye for determining VCD and % viability values among other nutrients and metabolites within the culture. The offline VCD values obtained from the bioanalyzer were correlated with the online permittivity generated using the capacitance probe, which generated a linear regression model that was used to predict the VCD throughout the course of the bioprocess.

The VCD throughout ST and production was monitored using both methods of detection. To provide additional confirmation that the capacitance values obtained were representative of the real-time VCD, the real-time VCD as obtained by correlating the permittivity and offline VCD was calculated and trended. Additionally, the three impedance parameters α, f_{c} and Δε were analyzed to assess if they provided additional insight about the cell culture when it was exposed to a series of failure modes throughout ST and production. Furthermore, to extend the capabilities of the capacitance probe, biovolume calculations were derived by applying the VCV calculation described above, further correlating the offline and online measurement readings.

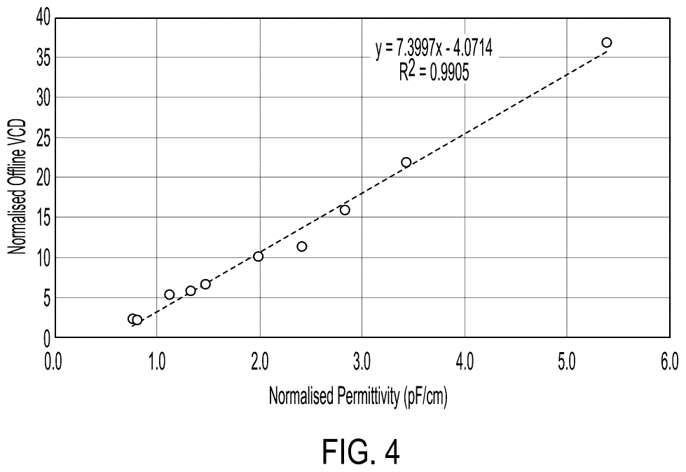

FIG. 4 shows the linear regression model linking the online permittivity readings to the off-line VCD measurements produced by the bioanalyzer throughout ST. The data within this graph was from a single bioreactor, A4, which was the control within the study and was not subjected to any failure mode excursions at any stage of the bioprocess. The values displayed were correlated so that each offline VCD timepoint was equivalent to the permittivity measurements from the online capacitance probe. The linear regression correlation model equation y=7.3997x−4.0714 was obtained, where x is the permittivity value, with an R² value of 0.9905. The R² value relates to the coefficient of determination or the proportion of variation between the dependent variable that is predictable from the independent one. The lowest possible R² value is 0 and the highest 1; therefore, the value attained from correlating the offline VCD to the permittivity, 0.9905, was high, confirming that the online permittivity measurements from the capacitance probe are accurate at predicting real-time VCD throughout ST.

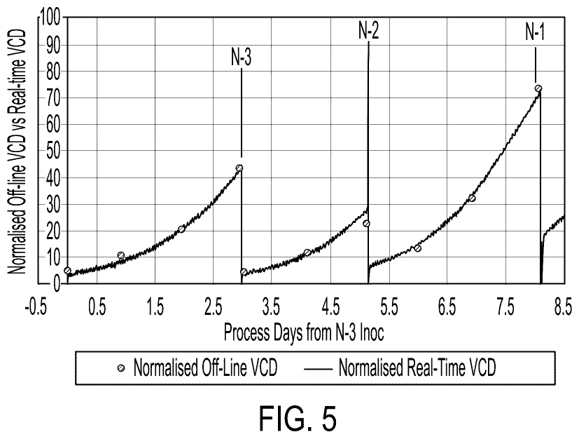

FIG. 5 depicts the calculated real-time VCD derived from the permittivity value, which was input into the correlation equation computed from the bivariate fit model in FIG. 4, in contrast with the offline VCD attained via the bioanalyzer. The continuous line (labeled Normalized Real-Time VCD) is representative of the real-time VCD, and the circles (labeled Normalied Off-Line VCD) are the offline VCD measurements. As the cell culture progressed throughout each stage of the ST process, the VCD steadily increased. The sudden drops in VCD seen within FIG. 5 are reflective of the transfers between phases of the ST bioprocess from N-3 to N-2 and lastly to N-1; within the scaled down model of a large-scale industrial bioprocess using 2L bioreactors, a series of cell dilutions were carried out to accurately reflect the large-scale process. Analyzing the real-time VCD compared to the offline VCD shows a strong correlation between the two variables as the circles of the offline VCD fit exactly onto the continuous line attained from the linear correlation equation and permittivity measurements from the capacitance probe. The transfer that occurred at the end of the N-2 phase of the ST process, outlined in FIG. 5 by a spike in the continuous line for the real-time VCD measurement at process day 5.0, is an artifact of improper conduct of the transfer and software management.

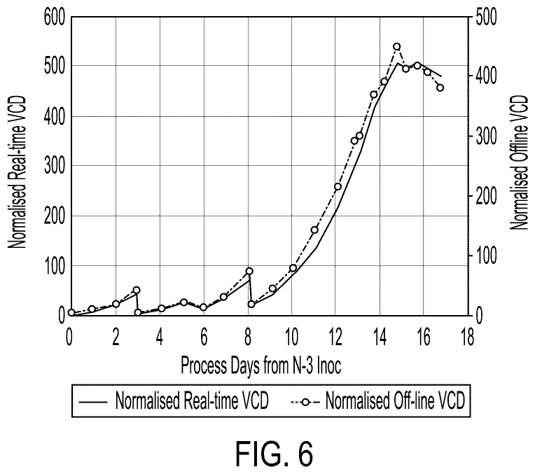

FIG. 6 illustrates the comparison between the offline VCD measurements obtained directly from the offline bioanalyzer and the real-time VCD derived from the permittivity value, which was input into the correlation equation computed from the bivariate fit model in FIG. 4. The dashed-line on FIG. 6 is representative of the offline VCD measurements obtained from the bioanalyzer, and the solid-line is the real-time VCD value calculated from the correlation linear regression equation and applied to all permittivity measurements for bioreactor A4. The linear regression model applied to the online permittivity measurements to create the real-time VCD values showed a strong correlation to the offline VCD measurements when analyzed throughout the ST phase of the bioprocess. However, as the cell culture progressed further along into the production stage of the bioprocess, discrepancies were seen between the two variables. It was observed around the day 10.5 mark of the entire bioprocess that the first obvious difference was evident between the real-time and offline measurements. From day 10.5 onwards, the difference between the two variables increased and the real-time VCD values obtained using the capacitance probe and linear regression equation had reduced accuracy.

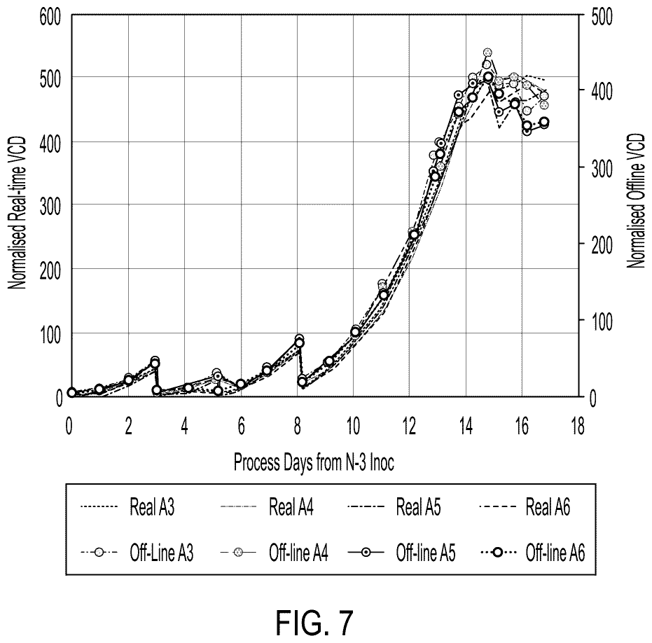

The offline VCD measurements were consistently lower across all four bioreactors, A3, A4, A5 and A6, in comparison to the real-time VCD, as shown in FIG. 7. The fluctuations present on the graph at about the day 14.5 mark are due to the failure modes inflicted on the cells. Metze et al., 2020a describes the strong link between online permittivity measurements for detecting VCD and VCV if cells are present within the exponential phase of growth (Metze et al., 2020, Bioprocess and Biosystems Engineering, 43:193-205). As the capacitance sensors overestimate the VCD values within the production phase of the bioprocess, it can be concluded that the linear correlation model equation is best applied when cells are in ST and are actively in the exponential phase of growth. As the real-time measurements obtained from the linear regression equation take into consideration the online permittivity, influential factors of permittivity must be considered to fully understand why this model had reduced accuracy at capturing real-time VCD throughout the production phase of growth.

The permittivity signal is influenced by the viable cell concentration and cell diameter, where larger cells are captured by a higher permittivity signal (Metze et al., 2020a). Additionally, the production phase features unique occurrences, such as nutrient feed additions, which change the cells' metabolic and morphological state and thus could have an influence on the permittivity measurements and the corresponding real-time VCD values. It has been referred to in the literature that the use of multivariate data analysis (MVDA) more accurately describes the interactions between various effects and influences cells experience, as it is less susceptible to small alterations, thus delivering a complex catalogue of statistically relevant data. Therefore, utilizing a MVDA model instead of a linear regression model across the production phase may reduce the requirement for manual sampling (Metze et al., 2020a).

Biovolume, or more specifically VCV, is an increasingly attractive parameter, as it combines offline cell diameter readings from a bioanalyzer with the online permittivity values obtained from the capacitance probe within the bioreactor. The correlation between VCV and permittivity could be an additional application of the measurement which is typically utilized to monitor cellular online viability and VCD. Δε is a direct correlation to the enclosed membrane volume, or cellular biovolume. As shown in FIG. 4, FIG. 5, FIG. 6, and FIG. 7, online permittivity measurements most accurately predict real-time VCD when the suspension is present in the lag/exponential phase of growth. When the cells enter apoptosis or the internal electric conductivity of the cells have been disrupted, permittivity becomes less useful for predicting real-time VCD even when calculated using a linear regression equation. Therefore, a calculation of interest to help link the CHO cell culture's online permittivity to the offline VCD values obtained via the bioanalyzer is VCV (4/3 πr³×VCD mm³/mL). Downey et al., 2014 states that this calculated equation accurately links cellular size and viable quantity to more accurately provide information about true biovolume as measured using capacitance probes.

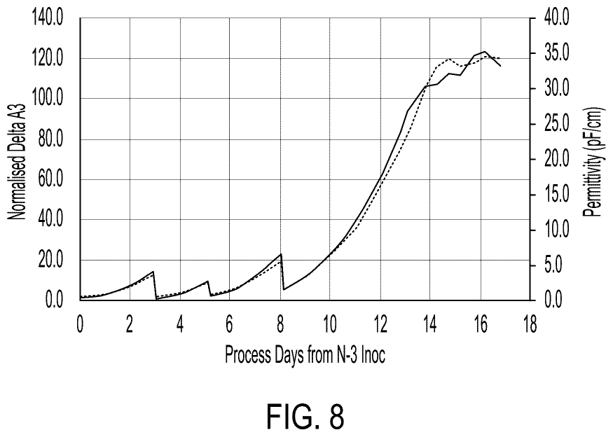

The evolution of biomass was investigated by harnessing the capabilities of Δε, which has been widely linked to biovolume measurements, particularly at high cellular growth rates, as it is a measure of the membrane-enclosed volume of the cells. At dual-frequency measurements, as exemplified in these examples, the biovolume has been shown to have a strong relationship with the permittivity increment and thus relates to the real-time biovolume of the culture. FIG. 8 clearly depicts the close relationship between these two parameters and thus confirms that the permittivity increment is an additional prediction of online biomass or indeed biovolume within suspension.

As seen with previous results in FIG. 6 and FIG. 7, the strong trend begins to waver as the production phase persists. However, applying more statistically relevant models such as MVDA techniques or Partial Least Squares (PLS) may increase the accuracy of these results. Two potential considerations when creating such a model would be, first, to create a model that can predict the changes in cell density, by cross analyzing with offline measurements. Second, to provide information on potential events that can result in a loss of the integrity of the membrane. Metze et al., 2020b applied Orthogonal Partial Least Squares (OPLS) modelling as an MVDA analysis technique to accommodate any variations in cell size distribution and metabolic changes within the culture due to nutrient feeding, an application that would not be possible with single frequency modelling (Metze et al., 2020b, Analytical and Bioanalytical Chemistry, 412:2089-2102). From this study it was observed that the viable cell concentration (VCC) possessed only a 10% relative error score compared to the single frequency model with an error score of 23%.

### Example 2. Evaluating the Ability of Capacitance Sensors to Detect Specific Failure Modes

Biomass is one of the most significant indicators of the progression of a bioprocess; however, the quantity of cells cultivated is not the only factor of interest, but equally their behavior. The biopharmaceutical industry frequently utilizes fed-batch bioprocesses to produce high yielding cell cultures in a reliable and predictable manner, by monitoring cellular progression using in-process controls such as pH, DO and temperature probes (Wong et al., 2005, Biotechnology and Bioengineering, 89(2):164-177). The future of this industry has already begun, with the drive for increased process monitoring controls, using continuous in situ probes which generate big data, that ultimately contributes to process improvement and optimization. However, current online sensor technologies still face significant challenges in accurately capturing the complex environments within the everchanging cellular suspensions (Konstantinov, 1996, Biotechnology and Bioengineering, 52(2):271-289). Alterations in bioreactor operating conditions have been directly linked to biomass evolution, and the effect of these perturbations on the cell culture in real-time has become of particular interest.

A capacitance probe was inserted into each of the four bioreactors to continuously monitor the effects of varying the bioreactor operating conditions on the impedance spectroscopy model parameters to assess biomass evolution. The model parameters of particular interest were f_{c}, α and Δε, which were obtained through fitting continuous measurements from the impedance data, such as permittivity, to the Cole-Cole conductivity model and a corresponding circuit model (Pacheco et al., 2021, Bioprocess and Biosystems Engineering, 44:1923-1934). These parameters have been linked to the physical state of the cell suspension. Alterations in these parameters throughout the evolution of the bioprocess have been attributed to biomass progression, concentration, morphology and cellular activity. However, the principle of impedance spectroscopy describes that these parameters are not just influenced by biomass progression, but also by alterations in bioreactor operating conditions. Previous studies such as one conducted by November et al. relate fluctuations in these parameters to alterations in aeration and agitation to the cell culture (November et al., 2000, Bioprocess Engineering, 23:473-477). A study by Soley et al. demonstrates how discrepancies in temperature, aeration and agitation affect the three parameters at a frequency of 10 kHz (Soley et al., 2005, Journal of Biotechnology, 118(4):398-405). Bioreactors are complex and dynamic systems which, even within tightly GMP controlled bioprocesses, can present various excursions (deviations from intended operating conditions), which can give rise to significant consequences in terms of biopharmaceutical production and deviation reporting. Therefore, ensuring that bioreactor conditions remain in a constant state of control is of major importance. To help achieve this, housing sensors that provide extensive real-time data can be used to ensure that early detection of these perturbations is captured. This can be achieved using capacitance probes.

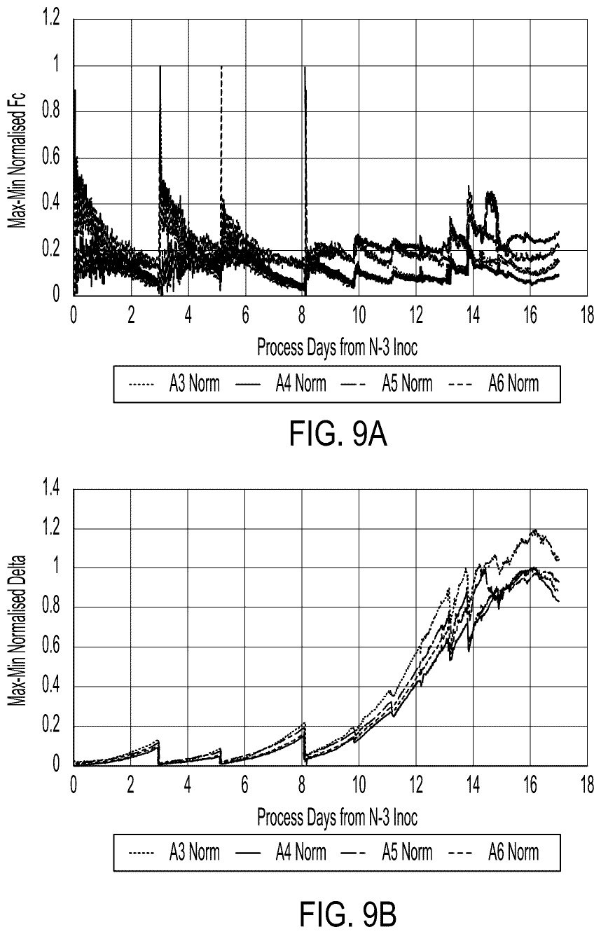

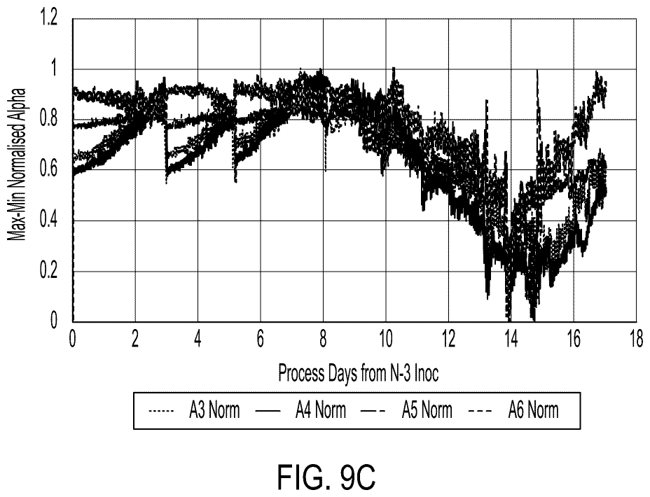

The inventors discovered that particular failure modes were of relevance to large-scale bioprocesses of interest, for example the production of Dupilumab, that had not been thoroughly investigated previously. Therefore, a method for real-time monitoring of failure modes of interest was developed. The exemplary failure modes tested were a high temperature excursion of five degrees Celsius in the N-3 phase of ST; a dissolved oxygen (DO) reduction of 25%; a missed dextrose feed; and an excess dextrose feed, all within the N phase. FIG. 9A, FIG. 9B and FIG. 9C show the evolution of each parameter f_{c}, α and Δε, respectively, throughout the course of the bioprocess encapsulating ST and production. F_{c} has been linked to the cell diameter and cellular dispersion throughout the suspension. Cell size distribution is linked to cell cycle, which is indicative of productivity (Lloyd et al., 2000). Typically, these parameters are attained using offline analytical tools which are subject to manual sample dilution ratios. Online monitoring captures the real-time information within bioreactors and therefore avoids any interferences encountered with offline sampling. F_{c} is the value obtained when the capacitance is halfway between the low and high frequency plateau. As the cell size increases, it results in an increase in the lower frequencies of the β-dispersion, thus causing a lower f_{c}. This can be observed in FIG. 9A where f_{c} appears higher during the ST phase of the bioprocess as the cells appear smaller. Additionally, during the exponential phase of cell growth, changes in cell size are typically minimal. F_{c} is also influenced by the conductivity of the suspension, which increases as the culture progresses throughout the bioprocess. This contributes to why f_{c} is a strong indication of the evolution of the process. A greater conductivity value means that there is an influx of ions within the suspension, and this causes a higher rate of polarization within the viable cells. Perturbations to the bioprocess observed in the N phase caused a decrease in permittivity, as the cells were disrupted; this in turn resulted in a lower relative volume of larger cells, which caused f_{c} to subsequently increase.

The Δε parameter is reflective of the biovolume measurements, particularly during the exponential phase of cell growth, as it is a measure of the membrane enclosed volume of the cells. FIG. 9B clearly depicts the progression of the cell culture throughout the bioprocess. As the cells get larger, the biovolume increases and thus Δε increases accordingly. This relationship between biovolume and Δε, however, begins to waver as the cells progress further into the N phase, as some cells start to become apoptotic. The increasing cell size and increasing viable biovolume are reflected in FIG. 9A and FIG. 9B, where f_{c} decreases with larger cell size and Δε increases as biovolume increases.

The Cole-Cole parameter α is used to factor in the relaxations observed to more accurately portray the dielectric dispersions present in a cellular suspension. α remains constant throughout the exponential phase of cell growth in the ST, as there are consistent excitations and relaxations to the cells, meaning that the relaxations observed are high. As the culture stabilizes in the N phase, the quantity of relaxations observed decrease until some cells begin to die and thus results in an increased quantity of observed relaxations. This is reflective of a more homogenous culture within the N phase. The α parameter helps to define the breadth of the relaxation time distribution within the 0-1 range. When α is set to 0, the Cole-Cole model simplifies to the Debye model. However, if α is greater than 0, it signifies that the relaxation process is spread across a broad frequency range. When the failure modes were inflicted to the bioprocess in the N phase, there was a clear increase in α due to the extension of the relaxation time spectrum.

### Example 3. High Temperature Excursion

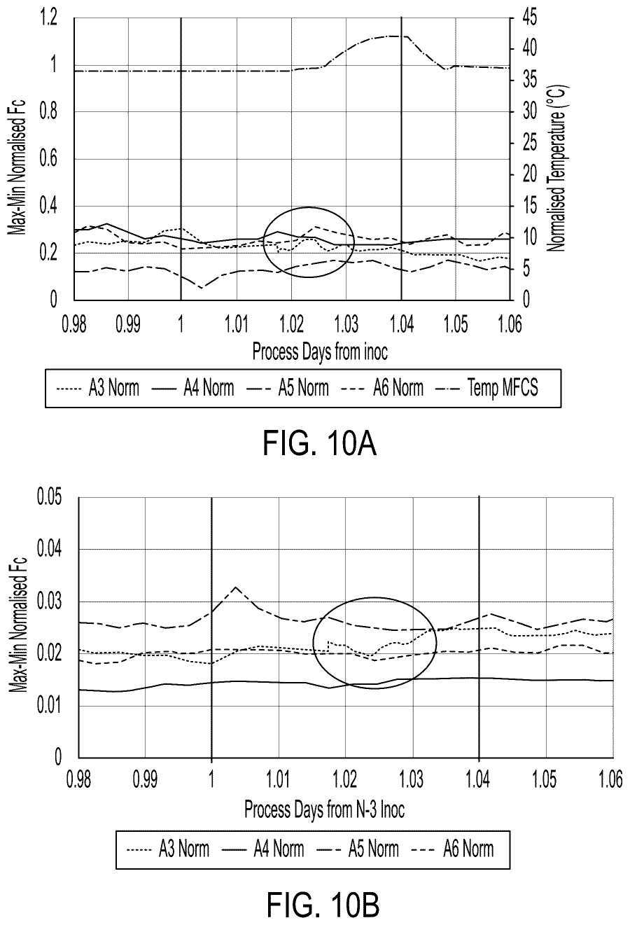

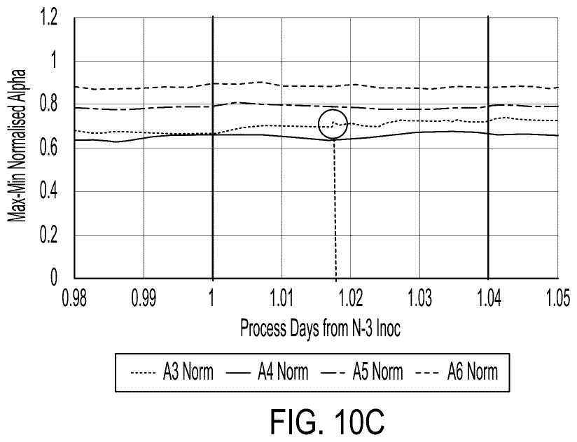

The high temperature excursion lasted a total of 40 minutes on process day one of ST; the excursion time frame can be seen as bolded vertical lines in FIG. 10A, FIG. 10B, and FIG. 10C, with each graph representing each of the three impedance parameters during that phase of the bioprocess. The cell biovolume or Δε at this stage of the bioprocess was low, as the culture had just entered ST the previous day (FIG. 10B). The bioreactor subjected to the high temperature excursion, A3, is shown on each graph. The temperature increase began on day 1.0 on each graph and theoretically carried on until day 1.04. However, as observed in FIG. 10A which illustrates the data from the multi fermenter control system (MFCS) captured from the in-process temperature probe located in each of the four bioreactors, the elevated temperature did last longer than 40 minutes. This is because the probes only captured the elevation in temperature much further into the duration of the excursion timeframe. Variations in each of the three parameters are illustrated by a circle on each of FIG. 10A, FIG. 10B, and FIG. 10C. The first indication of any sign of disruption to the cells appeared just before point 1.02 on each of the graphs, which, as shownn in FIG. 10A, was just when the temperature increase was detected by the MFCS system. The increase in f_{c} could have been a result of a morphological disturbance to the cells due to the temperature increase. As f_{c} is directly related to the morphology of the cell culture, any interference to cell morphology would be captured using this parameter. Equally, the brief decline in the Δε parameter could also be due to the temperature excursion to the cells. As Δε is directly related to the membrane enclosed volume of the cells, any perturbations that would impact the integrity of the cellular membrane would in turn be captured by Δε. Lastly, there is a slight spike in the α parameter at this exact timepoint in the bioprocess, which could be caused by the extension of the relaxation phenomena as a result of the temperature increase. However, there was a substantial amount of noise in each of the bioreactors detected by the capacitance probes, which could be related to the low biovolume within each bioreactor during that stage of the bioprocess. The capacitance probes may be able to more sensitively detect high temperature excursions in a culture with greater biomass, such as may be used in a commercial large-scale process.

It is important to note that the temperature excursion was not observed using the offline bioanalyzer. In contrast, the capacitance probe did show the ability to detect small perturbations within the bioprocess that could relate to the temperature rise using the three dielectric spectroscopy parameters.

### Example 4. Low DO and Excess Feed Excursion

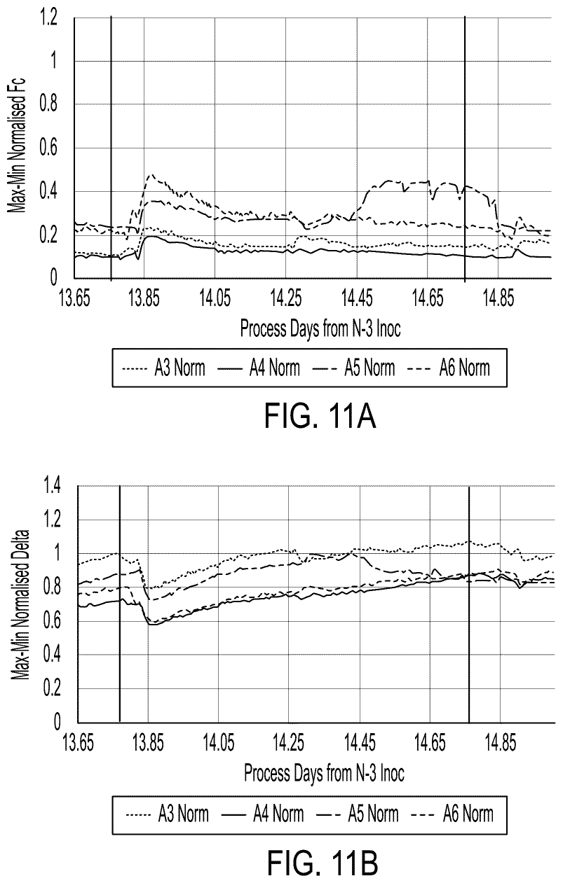

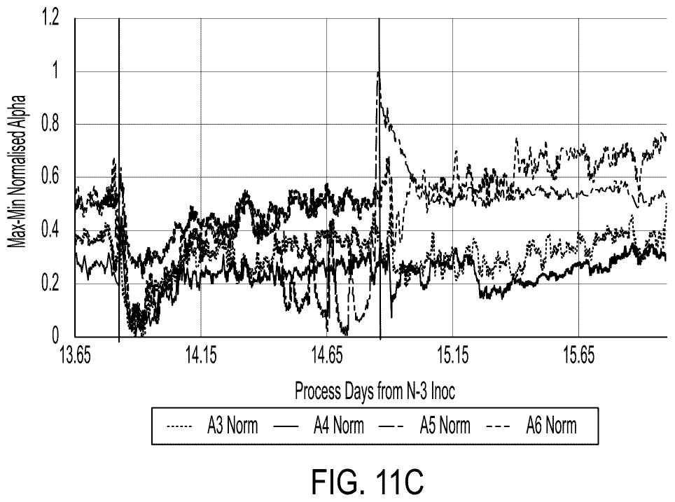

On day 5 of the production phase of the bioprocess, three additional failure mode scenarios were inflicted upon the cells: bioreactor A3 was subjected to low DO, A5 was subjected to a missed dextrose addition, and A6 was provided a double dextrose addition. The excursion time period commenced prior to the daily sampling using the offline bioanalyzer. This offline bioanalyzer would have been the sole analytical tool if the capacitance probes were not used to monitor the health of the culture. FIG. 11A, FIG. 11B, and FIG. 11C show each of the four bioreactors in this study during the twenty-four-hour excursion period, with A4 representing the control. The excursion timeframe is represented as bolded vertical lines on each of the graphs.

Firstly, with respect to bioreactor A3, it was expected that a low DO environment would significantly impact the cellular growth rate and viability, as oxygen uptake is crucial for many metabolic activities to occur within the aerobic cells and a lack of oxygen could potentially result in hypoxia, thus triggering apoptosis. However, upon analyzing FIG. 11A, FIG. 11B, and FIG. 11C, the low DO environment does not appear to have affected the suspension as drastically as anticipated. The first waver in each of the graphs just before the 13.85 day time point from N-3 inoculation is a result of a glucose addition after the first daily sample was taken. Additionally, this waver was repeated after the half daily sampling was carried out during the 14.25 day from N-3 inoculation. The impedance parameters f_{c}, α and Δε did not reflect a decline in cellular viability as a result of the low DO environment, and therefore the failure mode as implemented in this experiment was not captured using the capacitance sensor in bioreactor A3. A possible explanation as to why this failure mode was not detected might have been due to the time limitation of the excursion; twenty-four-hours may not have been long enough to have a lasting damaging effect on the viability of the cell culture. Furthermore, the DO setpoint might not have been impactful enough to elicit a change in the impedance parameters.

Bioreactor A6 was provided a double quantity of glucose during the primary sampling point of the excursion. It was hypothesized that alterations in nutrient availability would have corresponding implications in the β-dispersion parameters f_{c}, α and Δε, as they are a real-time glimpse into the physical and metabolic state of the cellular suspension. As observed in FIG. 9B, the continuous increase in Δε as the culture progresses throughout the bioprocess is reflective of the viable biovolume. However, it is also indicative of the constant increase in the cell diameter of the culture. As glucose was added to A6 at the first sampling point, there was a distinct drop in Δε, which could be indicative of a dilution of cell suspension or due to an increase in the osmolality driving a change in the cellular size. It can be concluded that it was not due to a drop in viability, as the cells recovered shortly afterwards, and therefore the physiological and morphological state of the cell culture was not significantly impacted by one excess glucose addition. This hypothesis was supported by the f_{c} parameter seen in FIG. 11A, as, although a spike was observed, representative of the double glucose addition, the change did not persist and the parameter levelled out as the culture evolved. As observed by Δε, in FIG. 11B, the effects of the glucose spike did not remain as the evolution of the cellular biovolume recovered after the initial dilution. Thus, it can be assumed that increases in f_{c} are directly linked to the nutrient additions to the cell culture, in addition to providing information about cell size evolution, directly correlating f_{c} to the internal metabolic activity of the culture.

### Example 6. Missed Dextrose Addition

Bioreactor A5 was subjected to nutrient limitations as the cells were deprived of glucose for twenty-four-hours. Therefore, at both sampling points during the excursion period, the cells were not provided a glucose feed. Although no glucose was added at the start of the excursion period as seen in FIG. 11A, day 13.85, there did appear to be an initial spike in f_{c}. A potential reason as to why this spike was observed could be due to the physical activity of taking the sample and the knock-on disruption to the cell culture that followed this activity. As observed for bioreactor A6, the multifrequency permittivity measurements f_{c} and Δε were reflective of nutrient availability when additional glucose was fed to the culture. F_{c} appeared to increase as the culture persisted and Δε was seen to decrease during this exact timeframe. F_{c} in relation to the nutrient limitation could be linked to the stress experienced by the cells in suspension. As reported for the excess glucose addition above, f_{c} was directly related to the metabolic activity and physiological state of the cells, therefore an increase in this parameter due to a nutrient limitation speaks to how permittivity measurements can provide extended insights into the physiological state of cell culture.

As there was no glucose addition during the second sampling timepoint of the excursion, the sudden decline of Δε seen in FIG. 11B cannot be due to a dilution effect. Instead, this decline is reflective of the biovolume reduction as the cells reduce in both size and viability. As f_{c} and Δε have an opposite relationship during this timeframe, it can be concluded that the cells are indeed reducing in size due to the glucose limitation. F_{c} is reflective of cell diameter and an increase in f_{c} is due to a reduction in cell size, whereas a reduction of Δε is directly reflective of a reduction in biovolume. Therefore, an increase in f_{c} and a decrease in Δε is an indication of a cell culture experiencing stress due to nutrient limitations.

F_{c} and α have an opposing relationship, which is seen in FIG. 11C. Both parameters began to diverge at the same timeframe of approximately 14.45 days from the N-3 inoculation. The Cole-Cole parameter α is reflective of the relaxations observed within the cellular suspension. During unfavourable environmental conditions to cells, such as a nutrient limitation that can trigger apoptosis, the α parameter is seen to vary largely due to an increased quantity of observed relaxations. The variation in α in response to the nutrient limitation could link it to the metabolic activity of the cell culture.

The above nutrient alterations were captured using the bioanalyzer, which detected a corresponding decline in cell size, viability, and fluctuations in glucose measurements after the twenty-four-hour excursion time had elapsed. The effect of reducing the availability of glucose to the cells was so severe that it caused them to produce lactate, which was captured using the bioanalyzer. As glucose is a major nutrient required for energy production and normal cellular biosynthesis to occur, a decline in its availability shifts cellular metabolism from glycolysis, which favors glucose, to one favoring lactate. Within mammalian cells, lactate production increases within stressed cells when the requirement for ATP and oxygen exceeds the availability of nutrients. The consequent increase in lactate after the excursion period validates the observed increase in f_{c} and decrease in Δε and α, linking these parameters to the metabolic and physiological state of the cell culture. Therefore, these examples demonstrate that capacitance probe measurements offered an early detection of cellular stress and an enhanced understanding of the metabolic and physiological state of the cell culture in response to cell culture conditions, providing real-time analysis which would not have been possible without them.

In summary, the dielectric spectroscopy impedance parameters f_{c}, Δε and α were used to detect the effects of different failure modes of interest on cells during the ST and N phases of a bioprocess. These parameters could be linked to specific failure modes and could be utilized as early process analytic technology (PAT) detection devices.

The key to attaining highly controlled and optimized processes is implementing real-time monitoring sensors into the bioprocess, as outlined by the FDA within the PAT Initiative. An increasingly promising analytical in-situ tool is dielectric spectroscopy, which offers non-invasive and continuous monitoring of cell culture evolution. This disclosure demonstrates that dielectric spectroscopy can accurately capture the link between permittivity and VCD, as outlined using a linear regression model. Additionally, various failure modes inflicted on the cells throughout the ST and N phase of the bioprocess were captured using the three dielectric spectroscopy impedance parameters f_{c}, Δε and α. The Cole-Cole model parameters presented different trends when under the influence of the same operating conditions, demonstrating their different implications with respect to cell morphology and physiology. The sensitivity and accuracy using the model parameters to detect the failure modes was seen to relate to the amount of biomass and the intensity of the failure mode. In particular, nutrient alteration excursions during the production phase resulted in significant fluctuations in the dielectric spectroscopy parameters, in particular f_{c} and Δε, showing that they can be reliably linked to specific failure modes.

The present invention is not to be limited in scope by the specific aspects described herein. Indeed, various modifications of the invention in addition to those described herein will become apparent to those skilled in the art from the foregoing description. Such modifications are intended to fall within the scope of the appended claims.

What is claimed is:

1. A method for characterizing a condition of a cell culture, comprising:
(a) measuring capacitance of a cell culture using a capacitance probe at two or more frequencies at two or more time points;
(b) using said capacitance measurement to calculate at least one impedance spectroscopy model parameter at said two or more time points; and
(c) using said at least one model parameter to characterize a condition of said cell culture.

2. The method of claim 1, wherein said cell culture comprises CHO cells.

3. The method of claim 1, wherein said cell culture comprises recombinant cells, wherein said recombinant cells express a protein selected from the group consisting of an antibody, an Fc-fusion protein, a receptor-Fc-fusion protein, and a fragment thereof.

4. The method of claim 1, wherein said cell culture comprises recombinant cells expressing Dupilumab.

5. The method of claim 1, wherein measuring capacitance of said cell culture includes measuring the permittivity of said cell culture.

6. The method of claim 1, wherein at least one of said two or more time points is from day 0 to day 20 of said cell culture, from day 1 to day 18, from day 3 to day 12, at day 0, at day 1, at day 2, at day 3, at day 4, at day 5, at day 6, at day 7, at day 8, at day 9, at day 10, at day 11, at day 12, at day 13, at day 14, at day 15, at day 16, at day 17, at day 18, at day 19, or at day 20.

7. The method of claim 1, wherein at least one of said two or more time points is during a cell culture phase selected from the group consisting of N-5, N-4, N-3, N-2, N-1, and N.

8. The method of claim 1, wherein at least one of said two or more time points is during a seed train phase of said cell culture.

9. The method of claim 1, wherein at least one of said two or more time points is during a production phase of said cell culture.

10. The method of claim 1, wherein at least one of said two or more frequencies is about 1 MHz.

11. The method of claim 1, wherein at least one of said two or more frequencies is about 20 MHz.

12. The method of claim 1, wherein said calculating comprises fitting said capacitance measurement to the Cole-Cole model.

13. The method of claim 1, wherein said at least one impedance spectroscopy model parameter is selected from the group consisting of the characteristic frequency (f_{c}), the alpha parameter (α), and the permittivity increment (Δε).

14. The method of claim 1, wherein said capacitance probe is embedded in a bioreactor.

15. The method of claim 1, wherein said condition is an operating condition of a bioreactor.

16. The method of claim 1, wherein said condition is a failure mode.

17. The method of claim 1, wherein said condition is selected from the group consisting of a temperature excursion, an insufficient dextrose feed, an excess dextrose feed, a dissolved oxygen excursion, and a pH excursion.
